(19) United States
(12) Patent Application Publication
(10) Pub. No.: US 2025/0381279 A1
(43) Pub. Date: Dec. 18, 2025

(54) CYTOTOXIN-CCL8 PEPTIDE CONSTRUCTS AND USES THEREOF

(71) Applicant: University of South Carolina, Columbia, SC (US)

(72) Inventors: Hippokratis Kiaris, Irmo, SC (US); Bernardo Chavez, Saint Matthews, SC (US)

(73) Assignee: University of South Carolina, Columbia, SC (US)

(21) Appl. No.: 19/238,843

(22) Filed: Jun. 16, 2025

Related U.S. Application Data
(60) Provisional application No. 63/660,798, filed on Jun. 17, 2024.

(51) Int. Cl. A61K 47/64; A61K 38/00; A61P 35/00; C07K 14/34; C07K 14/52

(52) U.S. Cl. CPC A61K 47/6415; A61P 35/00; C07K 14/34; C07K 14/523; A61K 38/00; C07K 2319/20

(57) ABSTRACT

Cytotoxin-CCL8 peptide constructs that include a cytotoxin linked to a CCL8 peptide and uses thereof. The cytotoxin can include a cytotoxic peptide such as a diphtheria toxin peptide. The constructs can be used to treat CCL8-related diseases, such as cancer, graft versus host disease (GVHD), microbial infections, and pulmonary fibrosis acute respiratory distress syndrome (ARDS), among others.

### CROSS-REFERENCE TO RELATED APPLICATIONS

Priority is claimed to U.S. Provisional Application 63/660,798, filed Jun. 17, 2024, which is incorporated herein by reference in its entirety.

### STATEMENT REGARDING FEDERALLY SPONSORED RESEARCH

This invention was made with government support under OIA1736150 awarded by the National Science Foundation. The government has certain rights in the invention.

### SEQUENCE LISTING

The instant application contains a Sequence Listing which has been submitted in XML format and is incorporated by reference in its entirety. The XML copy, created on Jun. 10, 2025, is named USPTO—250610—106479.049—SEQ_LIST.xml and is 9,470 bytes in size.

### FIELD OF THE INVENTION

The invention is directed to cytotoxin-CCL8 peptide constructs comprising a cytotoxin linked to a CCL8 peptide and uses thereof, such as for the treatment of cancer and other CCL8-related diseases.

### BACKGROUND

Chemoattractive cytokines (chemokines) are essential for the development and progression of various cancers (Nagarsheth et al, 2017; Ozga et al, 2021; Abdul-Rahman et al, 2024). Their activity targets both the cancer cells and the cells of tumor stroma including immune cells and fibroblasts, promoting cancer growth and metastatic spread, by producing antiapoptotic and mitogenic activity and by contributing to the transition of microenvironment from normal into pro-oncogenic proinflammatory microenvironment. CCL8 in particular, or alternatively designated as monocyte chemotactic protein-2 (MCP2), has been associated with the development of various cancers including breast cancer, by mechanisms involving the activation of tumor stroma through and the establishment of a gradient that favors dissemination of breast cancer cells, and the maintenance of a stem cell niche (Farmaki et al, 2016; Farmaki et al, 2020; Cassetta et al, 2019; Thomas et al, 2019; Zhang et al, 2020; Barbai et al, 2015; Lou et al, 2022).

In addition to cancer, CCL8 has also been associated with various immune system-related pathologies such as graft versus host disease (GVHD), microbial infections, and pulmonary fibrosis (Igarashi et al, 2021; Igarashi et al, 2014; Hori et al, 2008; Liu et al, 2013; Severa et al, 2014). More recently, a role for CCL8 has been proposed for the development of acute respiratory distress syndrome (ARDS) after SARS-COV-2 infection, while the beneficial effects of CCL8 inhibition have been described following LPS administration in mice (Thoutam et al, 2020; Blanco-Melo et al, 2020; Suhre et al, 2022; Naderi et al, 2022).

Agents and treatments that inhibit the activity of CCL8 are needed.

### SUMMARY OF THE INVENTION

One aspect of the invention is directed peptide constructs. The peptide constructs of the invention can comprise a cytotoxin linked to a CCL8 peptide.

In some versions, the cytotoxin comprises a cytotoxic peptide. In some versions, the cytotoxic peptide comprises a diphtheria toxin peptide. In some versions, the cytotoxic peptide comprises an amino acid sequence at least 80%, at least 85%, at least 90%, at least 95%, or at least 99% identical to SEQ ID NO:1.

In some versions, the CCL8 peptide comprises an amino acid sequence at least 80%, at least 85%, at least 90%, at least 95%, or at least 99% identical to SEQ ID NO:2.

In some versions, the cytotoxic peptide is linked to the CCL8 peptide via a peptide linker. In some versions, the peptide linker has a length from 1 to 30 amino acids. In some versions, a C-terminus of the cytotoxic peptide is linked to an N-terminus of the CCL8 peptide.

Another aspect of the invention is directed to methods of treating CCL8-related diseases. In some versions, the methods comprise administering a peptide construct of the invention or a nucleic acid configured to express a peptide construct of the invention to the subject in an amount effective to treat the CCL8-related disease. In some versions, the CCL8-related disease comprises one or more of cancer, graft versus host disease (GVHD), microbial infection, and pulmonary fibrosis acute respiratory distress syndrome (ARDS). In some versions, the CCL8-related disease comprises cancer. In some versions, the CCL8-related disease comprises breast cancer.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A and 1B. Structure of DTCCL8. FIG. 1A. Schematic depiction of the different components of the conjugate DTCCL8 (DT386-CCL8, top) or DT386 (bottom) are indicated. Linker is (G₄S₂) ₂ (SEQ ID NO:3). FIG. 1B. SDS-PAGE and western blot characterization of purified DT386-CCL8 expressed in E. coli. SDS-PAGE analysis (left), Western blot analysis using anti-6×His mAb (middle), or Western blot analysis using anti-hCCL8 mAb (right) of untransformed BL21 (lane 2), transformed BL21 with pET30a-DT386-CCL8, uninduced (lane 3), transformed BL21 with pET30a-DT386-CCL8 induced with 0.3 mM IPTG (lane 4), soluble proteins from lane 4 (lane 5), inclusion bodies isolated from lane 4 (lane 6), refolded and purified DT386-CCL8 via Nickel column chromatography. Arrow indicates the predicted DTCCL8 52.47 kDa band. Lane 1, molecular weight marker.

FIG. 2. SDS-PAGE analysis of DT386 expression and purification in E. coli. SDS-PAGE analysis of transformed BL21 with pET30a-DT386, uninduced (lane 2), transformed BL21 with pET30a-DT386 induced with 0.3 mM IPTG (lane 3), soluble proteins from lane 3 (lane 4), inclusion bodies isolated from lane 3 (lane 5), purified DTCCL8 (lane 6). Fractions of DT386 toxin purified by nickel affinity chromatography (lanes 7-9). Arrow indicates the predicted DT386 ˜42.94 kDa band. Lane 1, molecular weight marker.

FIG. 3. SDS-PAGE analysis of DTCCL8 and DT386 proteins under reducing and nonreducing conditions. Lane 1: molecular weight ladder. Lanes 2-3: DTCCL8 from the first batch, Lane 2 without DTT (non-reducing), Lane 3 with DTT (reducing). Lanes 4-5: DTCCL8 from the second batch, Lane 4 without DTT, Lane 5 with DTT. Lanes 6-7: DT386 protein, Lane 6 without DTT, Lane 7 with DTT. The expected molecular weight of DTCCL8 is 52.47 kDa, indicated by an arrow.

FIGS. 4A-4F. Cytotoxicity of DTCCL8 and the unconjugated DT386 toxin. Cell survival following treatment with DTCCL8 or DT386 at the concentrations indicated, in different cell lines after treatment for 3 days. Consistently DT386 produced moderately higher toxicity than DTCCL8 in cells cultured in vitro. BT549 showed resistance to DTCCL8 but after 6 days of treatment, sensitivity to DTCCL8 became apparent (FIG. 5).

FIG. 5. Cytotoxicity of DTCCL8 and unconjugated DT386 in BT549 and MDA-MB-231 cells. Cells were treated with DTCCL8 or DT386 at the indicated concentrations (concentration 0-5 μM) for 6 days. Cell viability was measured to assess toxin-induced cytotoxicity. DT386 shows consistently greater toxicity compared to DTCCL8 in both cell lines. Error bars represent mean±SEM.

FIGS. 6A-6D. DTCCL8 cytotoxicity depends on CCR5 expression. FIG. 6A. MDA-MB-231 cells transfected with CCR5 show increased sensitivity to DTCCL8. FIG. 6B. CHO-K1 cells transfected with CCR5 also show increased DTCCL8 cytotoxicity. For FIGS. 6A and 6B, conditions for each bar from left to right for each concentration is: Lipo (no DNA)+DTCCL8; CCR5+DTCCL8; Lipo (no DNA)+DT386; CCR5+DT386. FIG. 6C. CCR5 blockade by an inhibitory for CCR5 antibody reduces DTCCL8-induced cytotoxicity in MDA-MB-231 cells. FIG. 6D. A similar reduction is observed in BT549 cells following CCR5 antibody treatment. For transfection experiments, cells were treated with DTCCL8 or DT386 for 3 days following CCR5 transfection. For antibody blockade experiments, cells were pre-treated with anti-CCR5 antibody (clone 45531, 10 μg/mL) for 1 hour prior to toxin exposure and maintained in 7.5 μg/mL antibody throughout the 7-day treatment. Viability was assessed by XTT assay.

FIGS. 7A-7E. Internalization of DTCCL8 by breast cancer cells. FIG. 7A. Confocal microscopy images of BT549 cells treated with fluorescently labeled DTCCL8 (gray) at different concentrations. FIG. 7B. Quantification of fluorescence intensity in BT549 cells (left bars at each concentration) and MDA-MB-231 cells (right bars at each concentration) treated with 50 nM of labeled DTCCL8 or DT386, using the same fluorophore to assess non-specific uptake. FIG. 7C. Ab-mediated CCL8 neutralization abrogates DTCCL8 internalization. FIG. 7D. Quantification of results shown in FIG. 7C (control is left bar at each concentration; chimeric anti-CCL8 antibody (VL1B) is middle bar at each concentration; IgG1 is right bar at each concentration). FIG. 7E. Quantification of fluorescence intensity in BT549 and MDA-MB-231 cells treated with DT386 alone (left bars), DT386 plus anti-CCL8 antibody (VL1B) (middle bars), or DT386 plus IgG1 control antibody (right bars). Antibodies were used at a 10-fold molar excess relative to the toxin in FIGS. 7C, 7D, and 7E.

FIG. 8. Effect of DTCCL8 in total white blood cells (WBC), lymphocytes (LYM), monocytes (MON), and neutrophils (NEU) at 1.5 mg/kg. DTCCL8 was administered i.p. at t=0. Body weights are shown in the bottom panel.

FIG. 9. Effect of DTCCL8 on immune cell populations and body weight in an independent in vivo study. Mice were treated with DTCCL8 (1.5 mg/kg, intraperitoneally) at day 0. Blood counts were measured for total white blood cells (WBC), lymphocytes (LYM), monocytes (MON), and neutrophils (NEU) at the indicated time points. Body weight changes are shown in the bottom panel; each line represents an individual mouse. The arrow marks the time of DTCCL8 administration. Error bars represent mean±SEM. Statistical significance was determined using two-way ANOVA with Sidak's multiple comparisons test. **** P<0.0001, ns=not significant.

FIG. 10. Effect of DTCCL8 in the growth of Py-MT breast cancer isografts grown in wild type mice. Animals received DTCCL8 i.p. at 1.5 mg/kg at times indicated by vertical arrows. *P<0.05, students t test.

### DETAILED DESCRIPTION OF THE INVENTION

One aspect of the invention is directed peptide constructs. The peptide constructs of the invention can comprise a cytotoxin linked to a CCL8 peptide.

“Cytotoxin” is a term well known in the art and generally refers to agents or substances that can kill cells. Cytotoxins can comprise cytotoxic peptides, toxic metals, toxic chemicals (e.g., bleomycin, dactinomycin, daunorubicin, doxorubicin, epirubicin, idarubicin, mitomycin, mitoxantrone, plicamycin, valrubicin), microbe neurotoxins, radiation particles (radionuclides), and among other types of agents.

In some versions of the invention, the cytotoxin comprises a cytotoxic peptide. A large number of cytotoxic peptides are known in the art. Examples include diphtheria toxin, Toxin A of Clostridium difficile, Aurein 1.2, Magainin 2, Crotamine, TxID, Tv1, κ-PVIIA, human neutrophil peptide-1, Melittin, Tachyplesin I, Lactoferricin B, vesicular stomatitis virus G protein, snake venom cytotoxins, granulysin, perforin/granzyme B, Fas/Fas ligand, and sea anemone cytotoxic proteins, among others. See, e.g., Luan et al. 2021 (Luan X, Wu Y, Shen Y W, Zhang H, Zhou Y D, Chen H Z, Nagle D G, Zhang W D. Cytotoxic and antitumor peptides as novel chemotherapeutics. Nat Prod Rep. 2021 Jan. 1; 38 (1): 7-17), Ghandehari et al. 2015 (Ghandehari F, Behbahani M, Pourazar A, Noormohammadi Z. In silico and in vitro studies of cytotoxic activity of different peptides derived from vesicular stomatitis virus G protein. Iran J Basic Med Sci. 2015 January; 18 (1): 47-52), Vadevoo et al. (Vadevoo S M P, Gurung S, Lee H S, Gunassekaran G R, Lee S M, Yoon J W, Lee Y K, Lee B. Peptides as multifunctional players in cancer therapy. Exp Mol Med. 2023 June; 55 (6): 1099-1109), and Nhàn et al. 2023 (Nhàn NTT, Yamada T, Yamada K H. Peptide-Based Agents for Cancer Treatment: Current Applications and Future Directions. Int J Mol Sci. 2023 Aug. 18; 24 (16): 12931).

In some versions of the invention, the cytotoxic peptide comprises a diphtheria toxin peptide. “Diphtheria toxin peptide” as used herein refers to a peptide comprising an amino acid sequence of SEQ ID NO: 1 or sequence variants thereof that have the cytotoxic activity of a protein of SEQ ID NO:1. In various versions, the diphtheria toxin peptide can comprise an amino acid sequence at least 80% identical, at least 85% identical, at least 90% identical, at least 95% identical, at least 99% identical, or 100% identical to SEQ ID NO:1.

“CCL8 peptide” refers to a peptide comprising an amino acid sequence of SEQ ID NO:2 or sequence variants thereof that bind to a cognate receptor of CCL8 (a protein of SEQ ID NO:2). Exemplary cognate receptors of CCL8 include CCR1, CCR2, CCR3 and CCR5. In various versions of the invention, the CCL8 peptide can comprise an amino acid sequence at least 80% identical, at least 85% identical, at least 90% identical, at least 95% identical, at least 99% identical, or 100% identical to SEQ ID NO:2. In various versions of the invention, the CCL8 peptide can comprise an amino acid sequence at least 80% identical, at least 85% identical, at least 90% identical, at least 95% identical, at least 99% identical, or 100% identical to any 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, or 76 contiguous residues of SEQ ID NO:2.

The linkage between the cytotoxin and the CCL8 peptide can be via any peptide or chemical linkage.

In cases where the cytotoxin comprises a cytotoxic peptide, the linkage can be via any peptide-peptide linkage. A number of peptide-peptide linkages are known in the art. In some versions, the linkage occurs via the expression of the cytotoxic peptide and the CCL8 peptide as a fusion protein. The cytotoxic peptide and the CCL8 peptide can be linked in the fusion protein either directly or via a peptide linker. The cytotoxic peptide can be linked (either directly or via a peptide linker) to the CCL8 peptide at the N-terminus of the cytotoxic peptide or the C-terminus of the cytotoxic peptide. Similarly, the CCL8 peptide can be linked (either directly or via a peptide linker) to the cytotoxic peptide at the N-terminus of the CCL8 peptide or the C-terminus of the CCL8 peptide. In some versions, the cytotoxic peptide and the CCL8 peptide are linked via a chemical crosslinker. A large number of chemical crosslinkers suitable for crosslinking proteins are known in the art.

The peptide linker can be of any suitable length. “Length in this context refers to the number of amino acid residues between two moieties (e.g., a cytotoxic peptide and a CCL8 peptide). In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100, or more amino acids long. In some embodiments, the peptide linker is no more than about 100, 75, 50, 40, 35, 30, 25, 20, 19, 18, 17, 16, 15, 14, 13, 12, 11, 10, 9, 8, 7, 6, 5, or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. In some embodiments, the peptide linker is a flexible linker. Exemplary flexible linkers include any linkers disclosed in US 2025/0152759 A1, which is included herein by reference. Other suitable peptide linkers are provided in Klein et al. 2014 (Klein J S, Jiang S, Galimidi R P, Keeffe J R, Bjorkman P J. Design and characterization of structured protein linkers with differing flexibilities. Protein Eng Des Sel. 2014 October; 27 (10): 325-30) and Reddy Chichili et al. 2013 (Reddy Chichili V P, Kumar V, Sivaraman J. Linkers in the structural biology of protein-protein interactions. Protein Sci. 2013 February; 22 (2): 153-67).

Another aspect of the invention is directed to methods of treating CCL8-related diseases in a subject. The methods can comprise administering the peptide construct of the invention or a nucleic acid configured to express the peptide construct of the invention to the subject in an amount effective to treat the CCL8-related disease.

“CCL8-related disease” as used herein refers to any pathological condition mediated by CCL8 signaling. Many CCL8-related diseases are known in the art. These include cancers, such as breast cancer (Farmaki et al, 2016; Farmaki et al, 2020; Cassetta et al, 2019; Thomas et al, 2019; Zhang et al, 2020; Barbai et al, 2015; Lou et al, 2022); immune system-related pathologies such as graft versus host disease (GVHD), microbial infections, and pulmonary fibrosis (Igarashi et al, 2021; Igarashi et al, 2014; Hori et al, 2018; Liu et al, 2013; Severa et al, 2014); and acute respiratory distress syndrome (ARDS), particular after viral infection such as SARS-COV-2 infection (Thoutam et al, 2020; Blanco-Melo et al, 2020; Suhre et al, 2020; Naderi et al, 2022).

Exemplary cancers treatable with the methods of the invention include lung cancer (e.g., non-small cell lung cancer (NSCLC)), gastric cancer, colon cancer, heart cancer, neck cancer, breast cancer, melanoma (e.g., metastatic malignant melanoma), renal cancer (e.g. clear cell carcinoma), prostate cancer (e.g. hormone refractory prostate adenocarcinoma), bone cancer, pancreatic cancer, skin cancer, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, gastrointestinal carcinoid tumor, colorectal cancer, gastrointestinal stromal tumor, Leiomyosarcoma, T-cell lymphoma, environmentally induced cancers including those induced by asbestos, and combinations of such cancers.

The nucleic acid configured to express the peptide construct of the invention can comprise any type of nucleic acid, such as DNA or RNA, capable of being translated or transcribed and translated into one or more peptides. The nucleic acids may be configured so that the peptides are secreted from a cell in which they are produced. The delivery of nucleic acids in vivo for therapeutic applications is well known in the art.

In some versions, a nucleic acid configured to express the peptide construct is cloned into an expression vector. Methods commonly known in the art of recombinant DNA technology which can be used are described in Ausubel et al. (eds.), 1993, Current Protocols in Molecular Biology, John. Wiley & Sons, NY; and Kriegler, 1990, Gene Transfer and Expression, A Laboratory Manual, Stockton Press, N.Y.

A DNA encoding the peptide construct may be recombinantly engineered into a variety of host vector systems that also provide for replication of the DNA in large scale and contain the necessary elements for directing the transcription. The use of such a vector to transfect target cells in the patient will result in transcription of sufficient amounts of the peptide construct to affect a cellular process. For example, a vector can be introduced in vivo such that it is taken up by a cell and directs the transcription of the peptide construct. Such a vector can remain episomal or become chromosomally integrated, as long as it can be expressed to produce the desired peptide construct. Such vectors can be constructed by recombinant DNA technology methods standard in the art.

Vectors encoding the peptide construct can be plasmid, viral, or others known in the art, used for replication and expression in mammalian cells. Expression of the sequence encoding the peptide construct can be regulated by any promoter/enhancer sequences known in the art to act in mammalian, preferably human cells. Such promoters/enhancers can be inducible or constitutive. Such promoters include but are not limited to the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the viral CMV promoter, the human β-chorionic gonadotropin-6 promoter (Hollenberg et al., 1994, Mol. Cell. Endocrinology 106:111-119), etc. In one embodiment, cell type specific promoter/enhancer sequences may be used to promote the synthesis of the peptide construct in particular cells or tissue types.

Vectors for use in the practice of the invention include any eukaryotic expression vectors, including but not limited to viral expression vectors such as those derived from the class of retroviruses, lentiviruses, adenoviruses, or adeno-associated viruses.

Nucleic acids comprising a sequence encoding a peptide construct of the invention can be administered by way of gene delivery and expression into a host cell. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11 (5): 155-215.

In some embodiments, the nucleic acid encoding a peptide construct of the invention is directly administered in vivo, under conditions effective for production of the protein. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432).

In a specific embodiment, a viral vector that contains sequences encoding a peptide construct of the invention can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

In some embodiments of the invention, an adeno-associated viral vector may be used to deliver nucleic acid molecules that encode a peptide construct of the invention. The vector is designed so that, depending on the level of expression desired, a promoter and/or enhancer element of choice may be inserted into the vector.

Another approach to nucleic acid delivery involves transferring the nucleic acid to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host cell.

In some versions, the peptide construct of the invention is itself administered to the subject. The peptide construct of the invention and/or the nucleic acid configured to express same (collectively, “pharmaceutical agents”) can be administered in the form of a composition, such as a pharmaceutical composition. The pharmaceutical compositions can optionally include a pharmaceutically acceptable carrier, excipient, and/or stabilizer. The pharmaceutical compositions can be prepared by mixing a pharmaceutical agent having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose;

and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they should be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intra-arterial, intralesional or intraarticular routes, topical administration, inhalation or by sustained release or extended-release means. In some embodiments, the pharmaceutical composition is administered locally.

The pharmaceutical agents can be administered in accord with known methods, such as intravenous administration as a bolus or by continuous infusion over a period of time, by intramuscular, intraperitoneal, intracerobrospinal, subcutaneous, intravenous (i.v.), intra-articular, intrasynovial, intrathecal, oral, topical, or inhalation routes. A reconstituted formulation can be prepared by dissolving a lyophilized pharmaceutical agent described herein in a diluent such that the agent is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g. phosphate-buffered saline), sterile saline solution, Ringer's solution or dextrose solution, or aqueous solutions of salts and/or buffers.

In some embodiments, the pharmaceutical agents can be administered to the individual by subcutaneous (i.e. beneath the skin) administration. For such purposes, the pharmaceutical agents may be injected using a syringe. However, other devices for administration of the pharmaceutical agents are available such as injection devices; injector pens; auto-injector devices, needleless devices; and subcutaneous patch delivery systems.

In some embodiments, the pharmaceutical agents can be administered to the individual intravenously. In some embodiments, the pharmaceutical agent is administered to an individual by infusion, such as intravenous infusion. Infusion techniques for immunotherapy are known in the art (see, e.g., Rosenberg et al., New Eng. J. of Med. 319:1676 (1988)).

The individual can be an animal, such as a mammal or a human.

As used herein, “treatment” or “treating” is an approach for obtaining beneficial or desired results, including clinical results. For purposes of this invention, beneficial or desired clinical results include but are not limited to one or more of the following: alleviating one or more symptoms resulting from a condition, diminishing the extent of a condition, stabilizing a condition (e.g., avoiding or delaying the worsening of the disease), delaying or slowing the progression of a condition, ameliorating a condition state, decreasing the dose of one or more other medications required to treat the condition, increasing the quality of life, and/or prolonging survival. Also encompassed by “treatment” is a reduction of a pathological consequence of a condition. The methods of the invention contemplate any one or more of these aspects of treatment.

The term “therapeutically effective amount” used herein refers to an amount of an agent, a combination of agents, or a pharmaceutical composition comprising such agents sufficient to treat a specified disorder, condition, or disease, such as to ameliorate, palliate, lessen, and/or delay one or more of its symptoms.

The term “alignment” refers to a method of comparing two or more polynucleotides or polypeptide sequences for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22:4673-4680, 1994); CLUSTALV, (see, Larkin M. A., et al., CLUSTALW2, ClustalW and ClustalX version 2, Bioinformatics 23 (21): 2947-2948, 2007); Jotun-Hein, Muscle et al., MUSCLE: a multiple sequence alignment method with reduced time and space complexity, BMC Bioinformatics 5:113, 2004); Mafft, Kalign, ProbCons, and T-Coffee (see Notredame et al., T-Coffee: A novel method for multiple sequence alignments, Journal of Molecular Biology 302:205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The term “chromosomal integration” means the process whereby an incoming sequence is introduced into the chromosome of a host cell. The homologous regions of the transforming DNA align with homologous regions of the chromosome. Then, the sequence between the homology boxes can be replaced by the incoming sequence in a double crossover (i.e., homologous recombination). In some embodiments of the present invention, homologous sections of an inactivating chromosomal segment of a DNA construct align with the flanking homologous regions of the indigenous chromosomal region of the microbial chromosome. Subsequently, the indigenous chromosomal region is deleted by the DNA construct in a double crossover.

The term “consensus sequence” or “canonical sequence” refers to an archetypical amino acid sequence against which all variants of a particular protein or sequence of interest are compared. Either term also refers to a sequence that sets forth the nucleotides that are most often present in a polynucleotide sequence of interest. For each position of a protein, the consensus sequence gives the amino acid that is most abundant in that position in the sequence alignment.

The term “conservative substitutions” or “conserved substitutions” refers to, for example, a substitution of an amino acid with a conservative variant.

“Conservative variant” refers to residues that are functionally similar to a given residue. Amino acids within the following groups are conservative variants of one another: glycine, alanine, serine, and proline (very small); alanine, isoleucine, leucine, methionine, phenylalanine, valine, proline, and glycine (hydrophobic); alanine, valine, leucine, isoleucine, methionine (aliphatic-like); cysteine, serine, threonine, asparagine, tyrosine, and glutamine (polar); phenylalanine, tryptophan, tyrosine (aromatic); lysine, arginine, and histidine (basic); aspartate and glutamate (acidic); alanine and glycine; asparagine and glutamine; arginine and lysine; isoleucine, leucine, methionine, and valine; and serine and threonine.

The terms “corresponds to” or “corresponding to” refer to an amino acid residue or position in a first protein sequence being positionally equivalent to an amino acid residue or position in a second reference protein sequence by virtue of the fact that the residue or position in the first protein sequence aligns to the residue or position in the reference sequence using bioinformatic techniques, for example, using the methods described herein for preparing a sequence alignment. The corresponding residue in the first protein sequence is then assigned the position number in the second reference protein sequence.

The term “deletion,” when used in the context of an amino acid sequence, means a deletion in or a removal of one or more residues from the amino acid sequence of a precursor protein, resulting in a mutant protein having at least one less amino acid residue as compared to the precursor protein. The term can also be used in the context of a nucleotide sequence, which means a deletion in or removal of a nucleotide from the polynucleotide sequence of a precursor polynucleotide.

The term “DNA construct” and “transforming DNA” (wherein “transforming” is used as an adjective) are used interchangeably herein to refer to a DNA used to introduce sequences into a host cell or organism. Typically, a DNA construct is generated in vitro by PCR or other suitable technique(s) known to those in the art. In certain embodiments, the DNA construct comprises a sequence of interest (e.g., an incoming sequence). In some embodiments, the sequence is operably linked to additional elements such as control elements (e.g., promoters, etc.). A DNA construct can further comprise a selectable marker. It can also comprise an incoming sequence flanked by homology targeting sequences. In a further embodiment, the DNA construct comprises other non-homologous sequences, added to the ends (e.g., stuffer sequences or flanks). In some embodiments, the ends of the incoming sequence are closed such that the DNA construct forms a closed circle. The transforming sequences may be wildtype, mutant or modified. In some embodiments, the DNA construct comprises sequences homologous to the host cell chromosome.

In other embodiments, the DNA construct comprises non-homologous sequences. Once the DNA construct is assembled in vitro it may be used to: 1) insert heterologous sequences into a desired target sequence of a host cell; 2) mutagenize a region of the host cell chromosome (i.e., replace an endogenous sequence with a heterologous sequence); 3) delete target genes; and/or (4) introduce a replicating plasmid into the host.

A polynucleotide is said to “encode” an RNA or a polypeptide if, in its native state or when manipulated by methods known to those of skill in the art, it can be transcribed and/or translated to produce the RNA, the polypeptide, or a fragment thereof. The antisense strand of such a polynucleotide is also said to encode the RNA or polypeptide sequences. As is known in the art, a DNA can be transcribed by an RNA polymerase to produce an RNA, and an RNA can be reverse transcribed by reverse transcriptase to produce a DNA. Thus, a DNA can encode an RNA, and vice versa.

The term “expressed genes” refers to genes that are transcribed into messenger RNA (mRNA) and then translated into protein, as well as genes that are transcribed into types of RNA, such as transfer RNA (tRNA), ribosomal RNA (rRNA), and regulatory RNA, which are not translated into protein.

The terms “expression cassette” or “expression vector” refer to a polynucleotide construct generated recombinantly or synthetically, with a series of specified elements that permit transcription of a particular polynucleotide in a target cell. A recombinant expression cassette can be incorporated into a plasmid, chromosome, mitochondrial DNA, plasmid DNA, virus, or polynucleotide fragment. Typically, the recombinant expression cassette portion of an expression vector includes, among other sequences, a polynucleotide sequence to be transcribed and a promoter. In particular embodiments, expression vectors have the ability to incorporate and express heterologous polynucleotide fragments in a host cell. Many prokaryotic and eukaryotic expression vectors are commercially available. Selection of appropriate expression vectors is within the knowledge of those of skill in the art. The term “expression cassette” is also used interchangeably herein with “DNA construct,” and their grammatical equivalents.

“Gene” refers to a polynucleotide (e.g., a DNA segment), which encodes a polypeptide, and may include regions preceding and following the coding regions as well as intervening sequences (introns) between individual coding segments (exons).

The term “endogenous protein” refers to a protein that is native to or naturally occurring in a cell. “Endogenous polynucleotide” refers to a polynucleotide that is in the cell and was not introduced into the cell using recombinant engineering techniques. For example, a gene that was present in the cell when the cell was originally isolated from nature. A gene is still considered endogenous if the control sequences, such as a promoter or enhancer sequences that activate transcription or translation, have been altered through recombinant techniques.

The term “homologous sequences” as used herein refers to a polynucleotide or polypeptide sequence having, for example, about 100%, about 99% or more, about 98% or more, about 97% or more, about 96% or more, about 95% or more, about 94% or more, about 93% or more, about 92% or more, about 91% or more, about 90% or more, about 88% or more, about 85% or more, about 80% or more, about 75% or more, about 70% or more, about 65% or more, about 60% or more, about 55% or more, about 50% or more, about 45% or more, or about 40% or more sequence identity to another polynucleotide or polypeptide sequence when optimally aligned for comparison. In particular embodiments, homologous sequences can retain the same type and/or level of a particular activity of interest. In some embodiments, homologous sequences have between 85% and 100% sequence identity, whereas in other embodiments there is between 90% and 100% sequence identity. In particular embodiments, there is 95% and 100% sequence identity. “Homology” refers to sequence similarity or sequence identity. Homology is determined using standard techniques known in the art (see, e.g., Smith and Waterman, Adv. Appl. Math., 2:482, 1981; Needleman and Wunsch, J. Mol. Biol., 48:443, 1970; Pearson and Lipman, Proc. Natl. Acad. Sci. USA 85:2444, 1988; programs such as GAP, BESTFIT, FASTA, and TFASTA in the Wisconsin Genetics Software Package (Genetics Computer Group, Madison, Wis.); and Devereux et al., Nucl. Acid Res., 12:387-395, 1984). A non-limiting example includes the use of the BLAST program (Altschul et al., Gapped BLAST and PSI-BLAST: a new generation of protein database search programs, Nucleic Acids Res. 25:3389-3402, 1997) to identify sequences that can be said to be “homologous.” A recent version such as version 2.2.16, 2.2.17, 2.2.18, 2.2.19, or the latest version, including sub-programs such as blastp for protein-protein comparisons, blastn for nucleotide-nucleotide comparisons, tblastn for protein-nucleotide comparisons, or blastx for nucleotide-protein comparisons, and with parameters as follows: Maximum number of sequences returned 10,000 or 100,000; E-value (expectation value) of 1e-2 or 1e-5, word size 3, scoring matrix BLOSUM62, gap cost existence 11, gap cost extension 1, may be suitable. An E-value of 1e-5, for example, indicates that the chance of a homologous match occurring at random is about 1 in 10,000, thereby marking a high confidence of true homology.

The term “host cell” refers to a suitable host for an expression vector comprising a nucleic acid of the present invention. The host may comprise any organism, without limitation, capable of containing and expressing the nucleic acids or genes disclosed herein. The host may be prokaryotic or eukaryotic, single-celled or multicellular, including mammalian cells, plant cells, fungi, etc. Examples of single-celled hosts include cells of Escherichia, Salmonella, Bacillus, Clostridium, Streptomyces, Staphylococcus, Neisseria, Lactobacillus, Shigella, and Mycoplasma. Suitable E. coli strains (among a great many others) include BL21 (DE3), C600, DH5αF′, HB101, JM83, JM101, JM103, JM105, JM107, JM109, JM110, MC1061, MC4100, MM294, NM522, NM554, TGI, χ1776, XL1-Blue, and Y1089+, all of which are commercially available.

The term “identical” (or “identity”), in the context of two polynucleotide or polypeptide sequences, means that the residues in the two sequences are the same when aligned for maximum correspondence, as measured using a sequence comparison or analysis algorithm such as those described herein. For example, if when properly aligned, the corresponding segments of two sequences have identical residues at 5 positions out of 10, it is said that the two sequences have a 50% identity. Most bioinformatic programs report percent identity over aligned sequence regions, which are typically not the entire molecules. If an alignment is long enough and contains enough identical residues, an expectation value can be calculated, which indicates that the level of identity in the alignment is unlikely to occur by random chance.

The term “introduce” refers to, in the context of introducing a polynucleotide sequence into a cell, any method suitable for transferring the polynucleotide sequence into the cell. Such methods for introduction include but are not limited to protoplast fusion, transfection, transformation, conjugation, and transduction (see, e.g., Ferrari et al., Genetics, in Hardwood et al, (eds.), Bacillus, Plenum Publishing Corp., pp. 57-72, 1989).

The term “isolated” or “purified” means a material that is removed from its original environment, for example, a host cell if it is produced in a host cell, the natural environment if it is naturally occurring, or a cultivation broth if it is produced in a recombinant host cell cultivation medium. A material is said to be “purified” when it is present in a particular composition in a higher concentration than the concentration that exists prior to the purification step(s). For example, with respect to an element normally found in a cell, organism, or microbe (including a phage), such an element is “purified” when the final composition does not include some material from the original matrix.

The term “mutation” refers to, in the context of a polynucleotide, a modification to the polynucleotide sequence resulting in a change in the sequence of a polynucleotide with reference to a precursor polynucleotide sequence. A mutant polynucleotide sequence can refer to an alteration that does not change the encoded amino acid sequence, for example, with regard to codon optimization for expression purposes, or that modifies a codon in such a way as to result in a modification of the encoded amino acid sequence. Mutations can be introduced into a polynucleotide through any number of methods known to those of ordinary skill in the art, including random mutagenesis, site-specific mutagenesis, oligonucleotide directed mutagenesis, gene shuffling, directed evolution techniques, combinatorial mutagenesis, site saturation mutagenesis among others.

“Mutation” or “mutated” means, in the context of a protein, a modification to the amino acid sequence resulting in a change in the sequence of a protein with reference to a precursor protein sequence. A mutation can refer to a substitution of one amino acid with another amino acid, an insertion or a deletion of one or more amino acid residues. Specifically, a mutation can also be the replacement of an amino acid with a non-natural amino acid, or with a chemically-modified amino acid or like residues. A mutation can also be a truncation (e.g., a deletion or interruption) in a sequence or a subsequence from the precursor sequence. A mutation may also be an addition of a subsequence (e.g., two or more amino acids in a stretch, which are inserted between two contiguous amino acids in a precursor protein sequence) within a protein, or at either terminal end of a protein, thereby increasing the length of (or elongating) the protein. A mutation can be made by modifying the DNA sequence corresponding to the precursor protein. Mutations can be introduced into a protein sequence by known methods in the art, for example, by creating synthetic DNA sequences that encode the mutation with reference to precursor proteins, or chemically altering the protein itself. A “mutant” as used herein is a protein comprising a mutation.

The term “operably linked,” in the context of a polynucleotide sequence, refers to the placement of one polynucleotide sequence into a functional relationship with another polynucleotide sequence. For example, a DNA encoding a secretory leader (e.g., a signal peptide) is operably linked to a DNA encoding a polypeptide if it is expressed as a preprotein that participates in the secretion of the polypeptide. A promoter or an enhancer is operably linked to a coding sequence if it affects the transcription of the sequence. A ribosome binding site is operably linked to a coding sequence if it is positioned so as to facilitate translation. Generally, “operably linked” means that the DNA sequences being linked are contiguous, and, in the case of a secretory leader, contiguous and in the same reading frame.

The term “optimal alignment” refers to the alignment giving the highest overall alignment score.

The terms “percent sequence identity,” “percent amino acid sequence identity,” “percent gene sequence identity,” and/or “percent polynucleotide sequence identity,” with respect to two polypeptides, polynucleotides and/or gene sequences (as appropriate), refer to the percentage of residues that are identical in the two sequences when the sequences are optimally aligned. Thus, 80% amino acid sequence identity means that 80% of the amino acids in two optimally aligned polypeptide sequences are identical. The percent identities expressed herein with respect to a given named reference sequence are determined over the entire reference sequence, rather than only a portion thereof. Thus, an amino acid sequence at least about 80% identical to positions 1-42 of SEQ ID NO:4, for example, is at least about 80% identical to the entire sequence of positions 1-42 of SEQ ID NO:4, as opposed merely to subsequences thereof.

The term “plasmid” refers to a circular double-stranded (ds) DNA construct used as a cloning vector, and which forms an extrachromosomal self-replicating genetic element in some eukaryotes or prokaryotes, or integrates into the host chromosome.

A “promoter” is a polynucleotide sequence that functions to direct transcription of a downstream gene. In preferred embodiments, the promoter is appropriate to the host cell in which the target gene is being expressed. The promoter, together with other transcriptional and translational regulatory polynucleotide sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

The terms “peptide, “protein,” and “polypeptide” are used interchangeably herein. The 3-letter code as well as the 1-letter code for amino acid residues as defined in conformity with the IUPAC-IUB Joint Commission on Biochemical Nomenclature (JCBN) is used throughout this disclosure. It is also understood that a polypeptide may be coded for by more than one polynucleotide sequence due to the degeneracy of the genetic code.

“Recombinant”: A recombinant nucleic acid or polypeptide is one comprising a sequence that is not naturally occurring. A recombinant gene is a gene that comprises a recombinant nucleic acid sequence, is present within a cell in which it does not naturally occur, and/or is present in a different locus (e.g., genetic locus or on an extrachromosomal plasmid) within a particular cell than in a corresponding native cell. A recombinant cell (such as a recombinant microorganism) is one that comprises a recombinant nucleic acid, a recombinant gene, or a recombinant polypeptide. An example of a recombinant gene is a gene that has a coding sequence operably linked to a heterologous promoter.

The terms “regulatory segment,” “regulatory sequence,” or “expression control sequence” refer to a polynucleotide sequence that is operatively linked with another polynucleotide sequence that encodes the amino acid sequence of a polypeptide chain to effect the expression of that encoded amino acid sequence. The regulatory sequence can inhibit, repress, promote, or even drive the expression of the operably-linked polynucleotide sequence encoding the amino acid sequence.

The term “substantially identical,” in the context of two polynucleotides or two polypeptides refers to a polynucleotide or polypeptide that comprises at least 70% sequence identity, for example, at least 75%, at least 80%, at least 85%, at least 90%, at least 91%, at least 92%, at least 93%, at least 94%, at least 95%, at least 96%, at least 97%, at least 98%, or at least 99% sequence identity as compared to a reference sequence using the programs or algorithms (e.g., BLAST, ALIGN, CLUSTAL) using standard parameters.

“Substantially purified” means molecules that are at least about 60% free, preferably at least about 75% free, about 80% free, about 85% free, and more preferably at least about 90% free from other components with which they are naturally associated. As used herein, the term “purified” or “to purify” also refers to the removal of contaminants from a sample.

“Substitution” means replacing an amino acid in the sequence of a precursor protein with another amino acid at a particular position, resulting in a mutant of the precursor protein. The amino acid used as a substitute can be a naturally-occurring amino acid, or can be a synthetic or non-naturally-occurring amino acid.

The term “transformed” or “stably transformed” cell refers to a cell that has a non-native (heterologous) polynucleotide sequence integrated into its genome or as an episomal plasmid that is maintained for at least two generations.

“Vector” refers to a polynucleotide construct designed to introduce polynucleotides into one or more cell types. Vectors include cloning vectors, expression vectors, shuttle vectors, plasmids, cassettes and the like.

“Wild-type” means, in the context of gene or protein, a polynucleotide or protein sequence that occurs in nature. In some embodiments, the wild-type sequence refers to a sequence of interest that is a starting point for protein engineering.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### A Chimeric Diphtheria Toxin-CCL8 Cytotoxic Peptide for Breast Cancer Management

### Summary

Deregulation of chemokine CCL8 expression is common in various malignancies and other pathologies and plays a causative role in disease progression. However, despite its acknowledged role of CCL8 in pathology, inhibition of its activity does not represent a strategy of choice for cancer management because its function overlaps with the function of other structurally related chemokines, and its activity is mediated by more than one receptor. To overcome this limitation, we hypothesized that ablation of CCL8 cellular targets, as opposed to disruption of CCL8 activity, may be more advantageous. Therefore, we developed DTCCL8, a chimeric cytotoxic peptide that delivers diphtheria toxin into cells expressing CCL8 receptors which are overexpressed in both the cells of tumor microenvironment and the cancer cells. The specificity of this peptide was confirmed in vitro by testing the cytotoxic activity of breast cancer cells overexpressing CCR5, a major CCL8 receptor, and by a neutralizing anti-CCL8 antibody we developed. In vivo, DTCCL8 transiently reduced lymphocytes in blood, and its anticancer activity was confirmed in mouse breast cancers triggered by the polyoma middle T oncogene. These findings suggest that DTCCL8 can be used as a prototype for the development of a novel class of breast cancer therapeutics, targeting chemokine targets instead of inhibiting their activity. These cytotoxic peptides may also be useful for the management of cancer and of other immune system-associated pathologies.

### Introduction

Chemoattractive cytokines (chemokines) are essential for the development and progression of various cancers (Nagarsheth et al, 2017; Ozga et al, 2021; Abdul-Rahman et al, 2024). Their activity targets both cancer cells and stromal cells including immune cells and fibroblasts, promoting cancer growth and metastatic spread, by producing antiapoptotic and mitogenic activity and by contributing to the transition of microenvironment from normal into pro-oncogenic proinflammatory microenvironment. CCL8 in particular, or alternatively designated as monocyte chemotactic protein-2 (MCP2), has been associated with the development of various cancers including breast cancer, by mechanisms involving the activation of tumor stroma and establishment of a gradient that favors dissemination of breast cancer cells, and the maintenance of a stem cell niche (Farmaki et al, 2016; Farmaki et al, 2020; Cassetta et al, 2019; Thomas et al, 2019; Zhang et al, 2020; Barbai et al, 2015; Lou et al, 2022).

In addition to cancer, CCL8 has also been associated with various immune system-related pathologies such as graft versus host disease (GVHD), microbial infections, and pulmonary fibrosis (Igarashi et al, 2021; Igarashi et al, 2014; Hori et al, 2018; Liu et al, 2013; Severa et al, 2014). More recently, a role for CCL8 has been proposed for the development of acute respiratory distress syndrome (ARDS) after SARS-COV-2 infection, while the beneficial effects of CCL8 inhibition have been described following LPS administration in mice (Thoutam et al, 2020; Blanco-Melo et al, 2020; Suhre et al, 2020; Naderi et al, 2022).

The evidence linking CCL8 with cancer development advocate for the development of CCL8-based therapeutics to inhibit CCL8 activity. Nonetheless, no major attempts to develop CCL8-targeting moieties exist so far, and only few efforts to target similar chemokines have been implemented, with limited success. The reason behind this gap is probably related to a combination of factors. For example, CCL8 is highly similar to other chemokines of the same cytokine cluster, such as CCL7 and CCL11, exhibiting redundancy in their activities (Farmaki et al, 2016; Hughes and Nibbs, 2018). Thus, inhibition of CCL8 would be inadequate in effectively suppressing the corresponding activities that lead to pathology. In addition, each of these chemokines recognizes more than one receptor. For example, CCL8 is an agonist for C-C chemokine receptor type 2 (CCR2), CCR3, CCR5, therefore, targeting a single receptor instead of the ligand would be insufficient for the prevention of the corresponding activity (Blaszczyk et al, 2000; Wang et al, 2013; Wang et al, 2014; Sokol et al, 2018).

To overcome these limitations, we hypothesized that instead of targeting either CCL8 or its receptors, a strategy of choice would be to target the cellular targets of CCL8, irrespectively of the specific receptor types that are expressed in the corresponding cell types. The premise of this hypothesis is that the receptors for CCL8, especially of CCR2 and CCR5, are overexpressed in breast cancers, and thus their presence sensitizes cells against specific receptor-targeting moieties (Fang et al, 2016; Fang et al, 2021; Wang et al, 2021; Jiao et al, 2018; Velasco-Velazquez et al, 2013). In addition, responsiveness to CCL8 is a feature of the tumor microenvironment, expanding the roster of the prooncogenic targets in tumors (Farmaki et al, 2016; Farmaki et al, 2020; Cassetta et al, 2019). To test this hypothesis, we developed the DTCCL8 prototype, which is chimeric peptide that comprises CCL8 linked to the highly cytotoxic diphtheria toxin (DT). Similar strategies have been employed for the management of different cancers with encouraging results. In most of the cases the cytotoxic peptide included DT conjugated to IL-2, IL-3 or the single chain fragment variable (scFV) against CD19 antigen (Alkharabsheh and Frankel, 2018; Wang et al, 2020; Zheng et al, 2019; Shafiee et al, 2019) and in the case of the IL-3 conjugate, the corresponding drug, designated Tagraxofusp, has received approval for the treatment of hematologic malignancies (Syed 2019). Here we show that DTCCL8 effectively targets cells by a manner that depends on CCR5 expression, and that its activity is antagonized by a neutralizing anti-CCL8 antibody. In vivo, DTCCL8 inhibited the growth of mouse breast cancers that were induced by the polyoma middle T (PymT) oncogene.

### Results

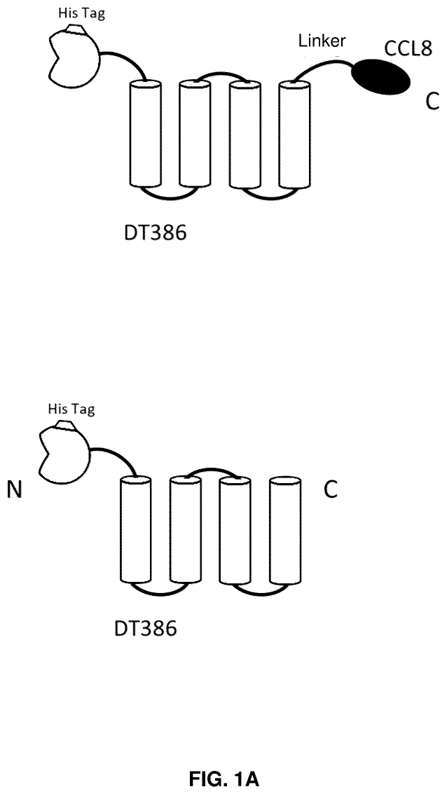

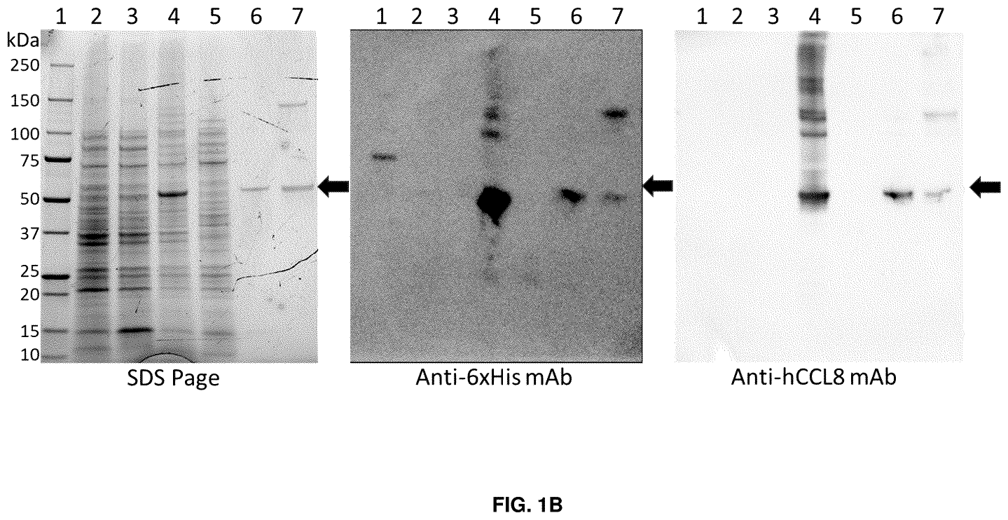

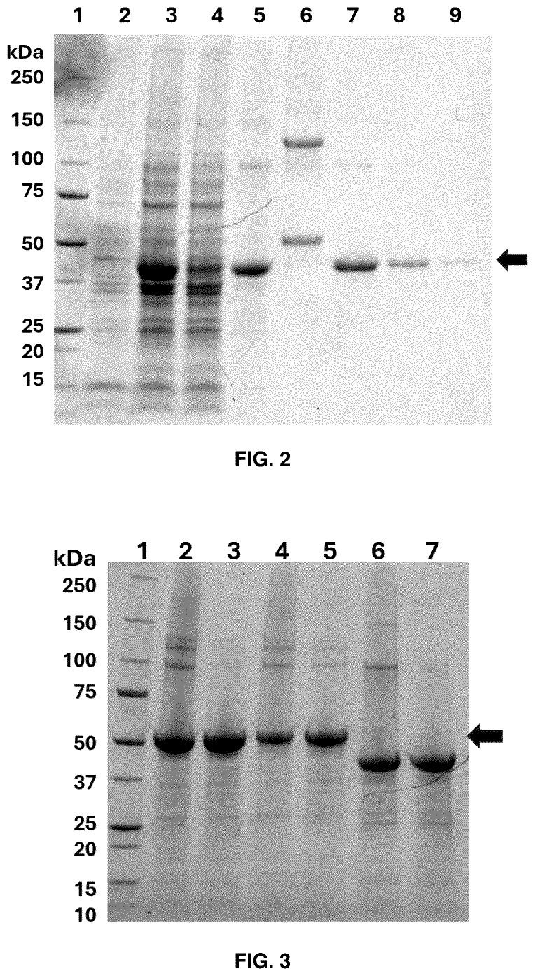

Development of DTCCL8. Using recombinant DNA technology, we generated a pET30a-based plasmid expressing a chimeric peptide (SEQ ID NO:5) comprising diphtheria toxin (DT386) (SEQ ID NO:1) and the full length human CCL8 (SEQ ID NO:2), separated by a dimer of 4×Glycine-Ser linker (SEQ ID NO:3) (FIG. 1A). A His-Tag was introduced in the N-terminus of the DT386 to enable identification. As shown in FIGS. 1B, 2, and 3, the purified peptide (DTCCL8) had the expected size of about 52.5 kDa, and was detectable by both an immunoblot probed by anti-His antibody and an antibody against CCL8 that we previously developed (Naderi et al, 2022). Under reducing conditions, a single band was seen (FIG. 1B, lane 6) while following refolding, a larger band, likely corresponding to a DTCCL8 multimer, appeared (FIG. 1B, lane 7). Further characterization of DT386 expression and purification, including SDS-PAGE profiles and analysis under reducing and non-reducing conditions, is shown in FIGS. 2 and 3.

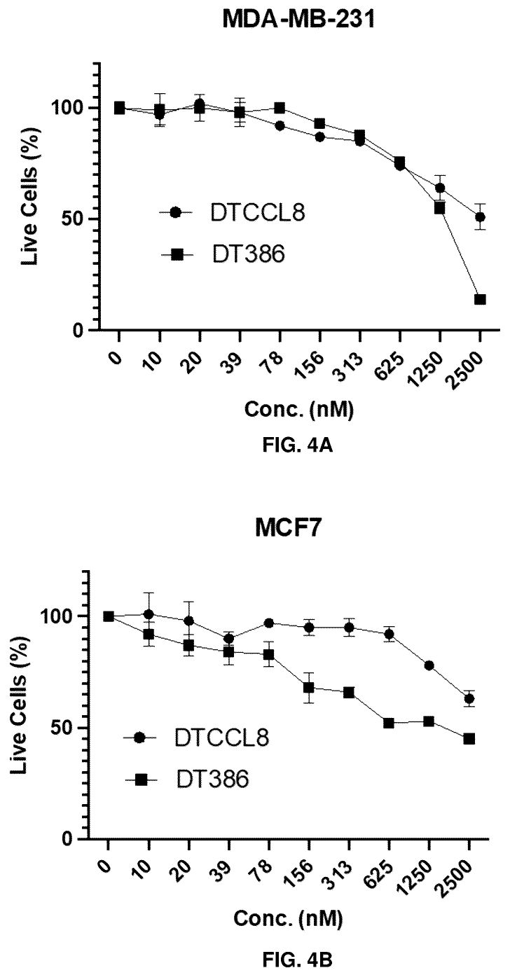

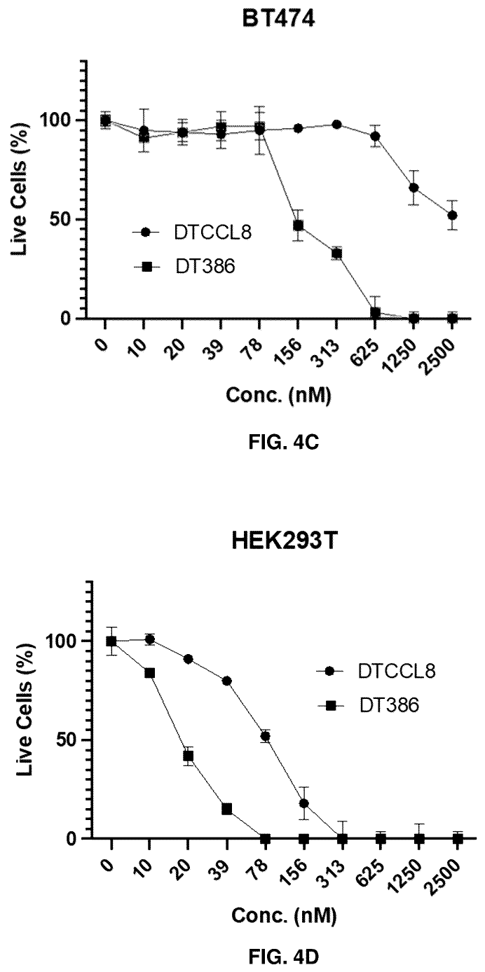

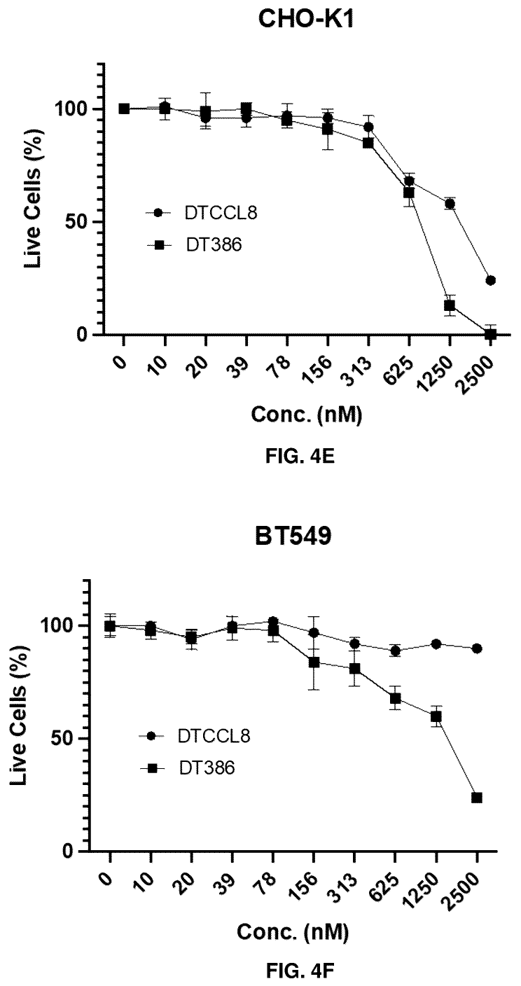

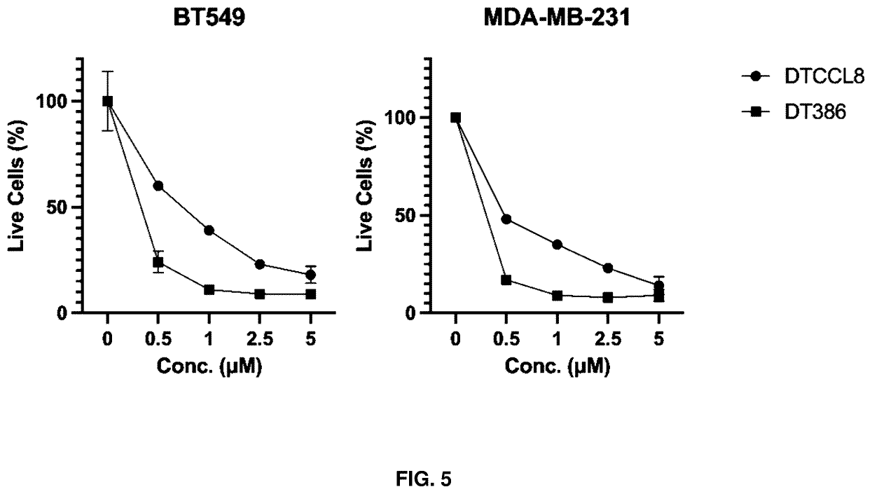

DTCCL8 activity is sensitized by CCR5 expression and is antagonized by anti-CCL8 treatment. Initially, we confirmed that DTCCL8 can be internalized by cells using time-lapse confocal imaging of a HEK293T cell treated for 24 hours with fluorescently labeled DTCCL8 (Supplementary video V1) Then, we assessed the cytotoxicity of DTCCL8 in comparison with equimolar amount of unconjugated DT386 toxin in different cancer cell lines. DTCCL8 was consistently slightly less toxic than DT386 at equimolar concentrations (FIGS. 4A-4F and 5). While DTCCL8 is designed for receptor-mediated targeting, its reduced toxicity at high concentrations may reflect variable or low expression of CCL8 receptors in monoculture or potential receptor saturation. In contrast, DT386 may enter cells through receptor-independent mechanisms, particularly at high concentrations, as previously reported for truncated diphtheria toxins (Zhang et al., 2010). Consistent with this, DT386 administered at 1.5 mg/kg in mice was acutely toxic, leading to death in treated animals, whereas DTCCL8 at the same dose was well tolerated and used in subsequent in vivo studies (see below).

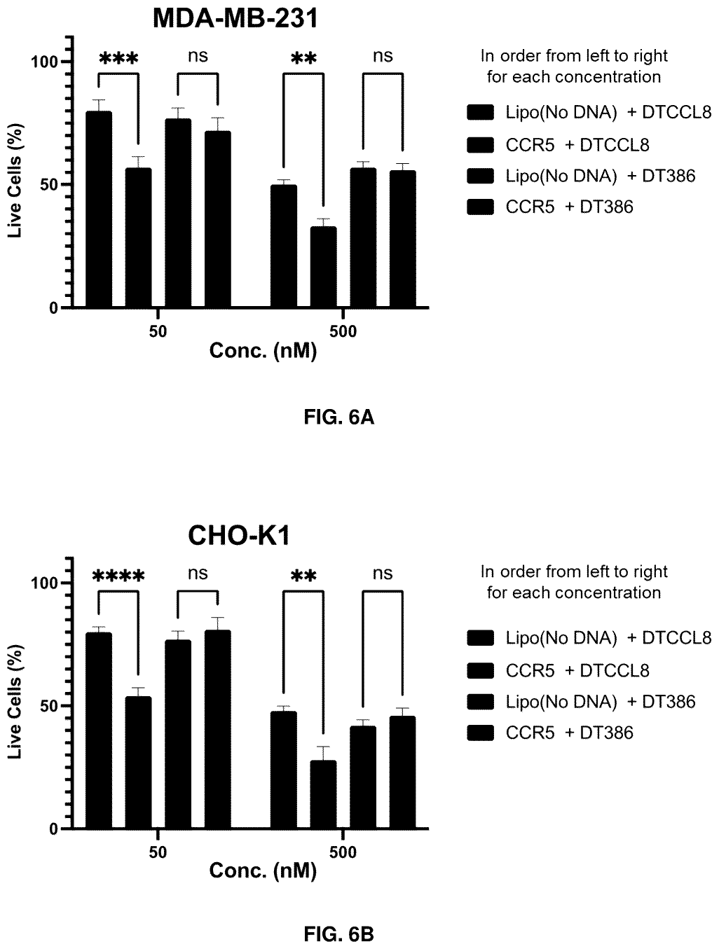

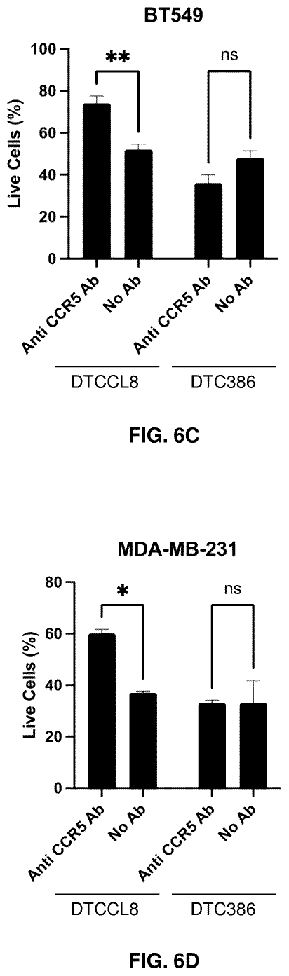

Next, we transiently overexpressed CCR5, the major CCL8 receptor, in MDA-MB-231 and CHO-K1 cells and explored the toxicity of DTCCL8 and DT386. As shown in FIGS. 6A and 6B, expression of CCR5 sensitized both cell types against DTCCL8 conferring cytotoxicity at concentrations at which untransfected cells are resistant to the drug. In contrast, DT386 toxicity was unaffected by CCR5 expression, indicating that DT386 does not benefit from CCR5-mediated uptake. These results confirm that DTCCL8 cytotoxicity is mediated specifically through CCL8 receptor engagement. To assess the role of CCR5 in DTCCL8 activity, MDA-MB-231 and BT549 cells were pre-treated with a CCR5-blocking antibody (clone 45531) prior to exposure to 500 nM DTCCL8 or DT386. The antibody was maintained throughout the 7-day treatment period. DTCCL8-induced cytotoxicity was reduced in both cell lines following CCR5 blockade, while DT386 toxicity was unaffected (FIGS. 6C and 6D).

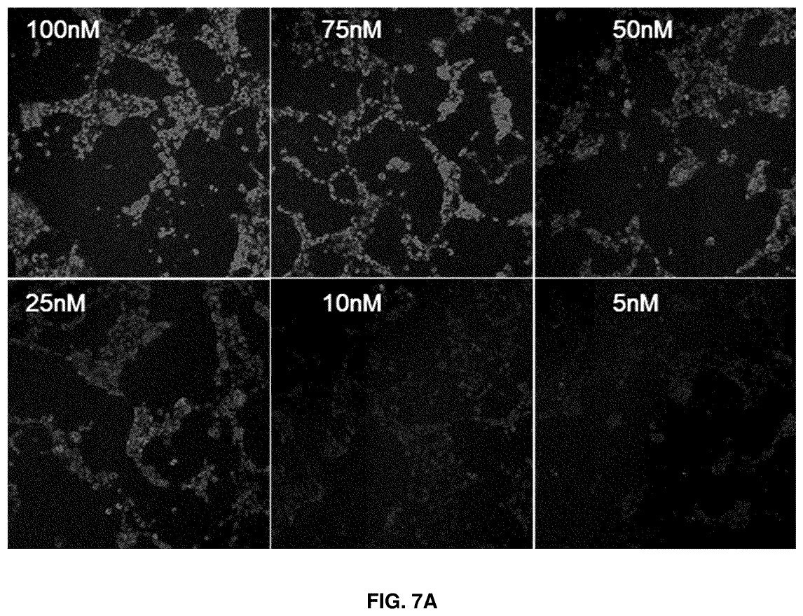

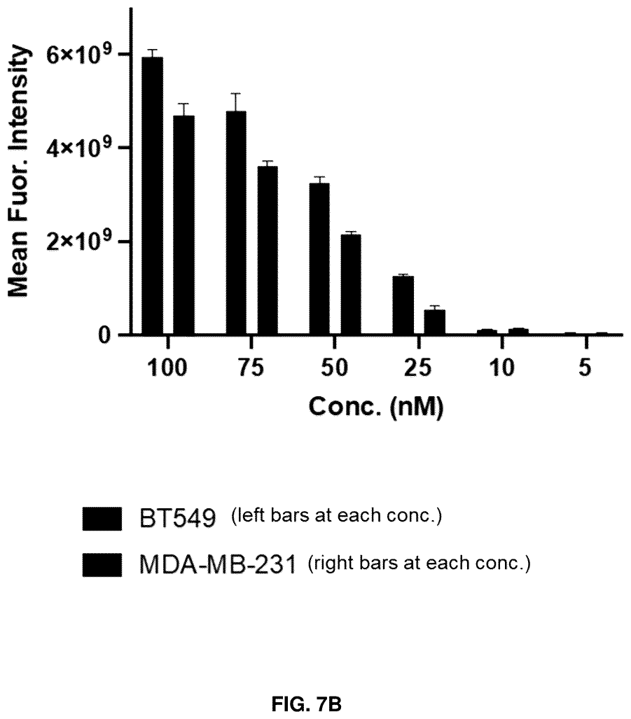

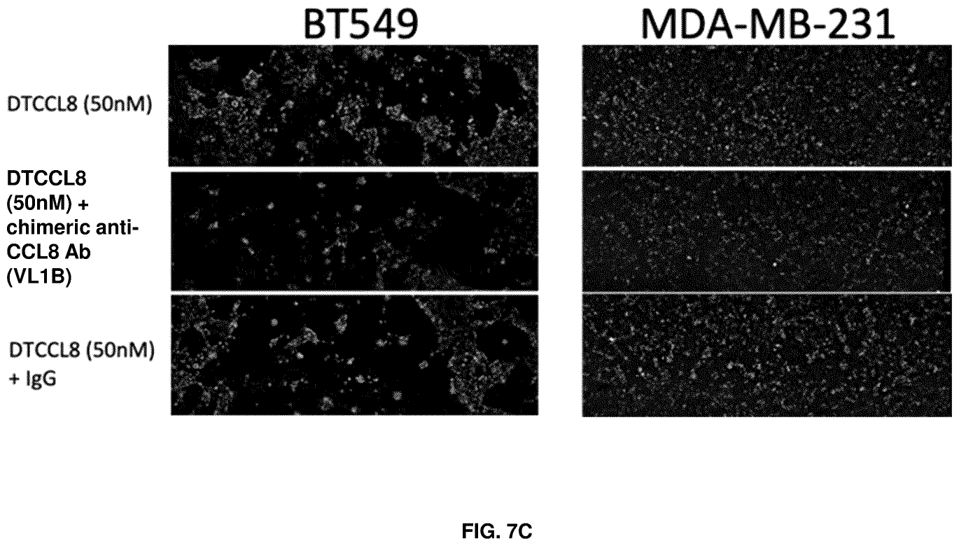

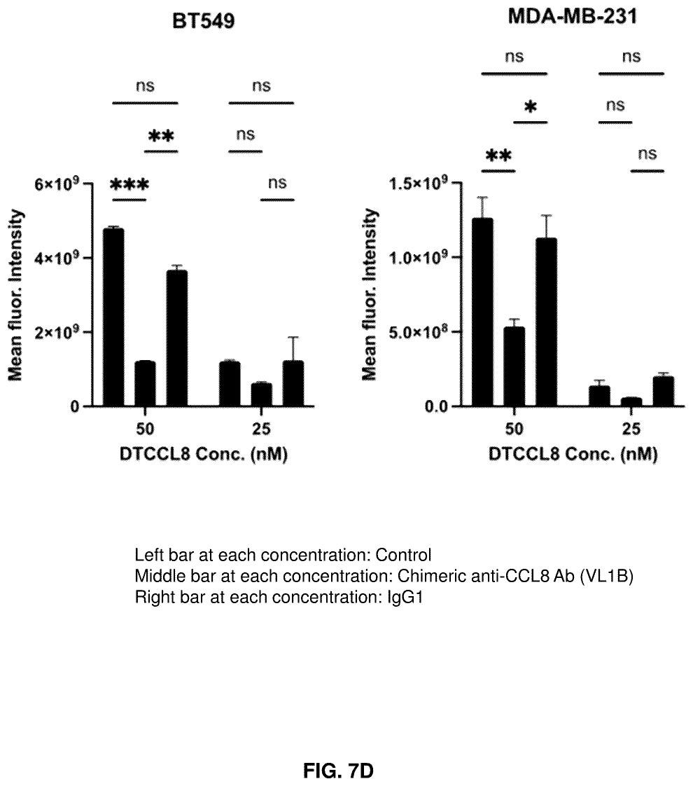

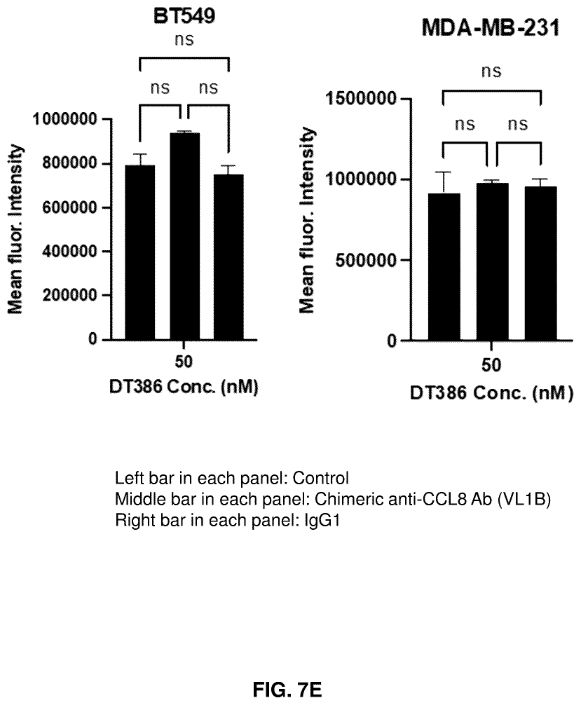

To evaluate receptor-mediated uptake, we assessed internalization of fluorescently labeled DTCCL8 and DT386 in breast cancer cells. Both MDA-MB-231 and BT549 cells internalized DTCCL8 (FIGS. 7A and 7B), but uptake was significantly reduced when co-treated with a neutralizing anti-CCL8 antibody, while control IgG1 had no effect (FIGS. 7C and 7D). Internalization of DT386 was not affected by either antibody (FIG. 7E).

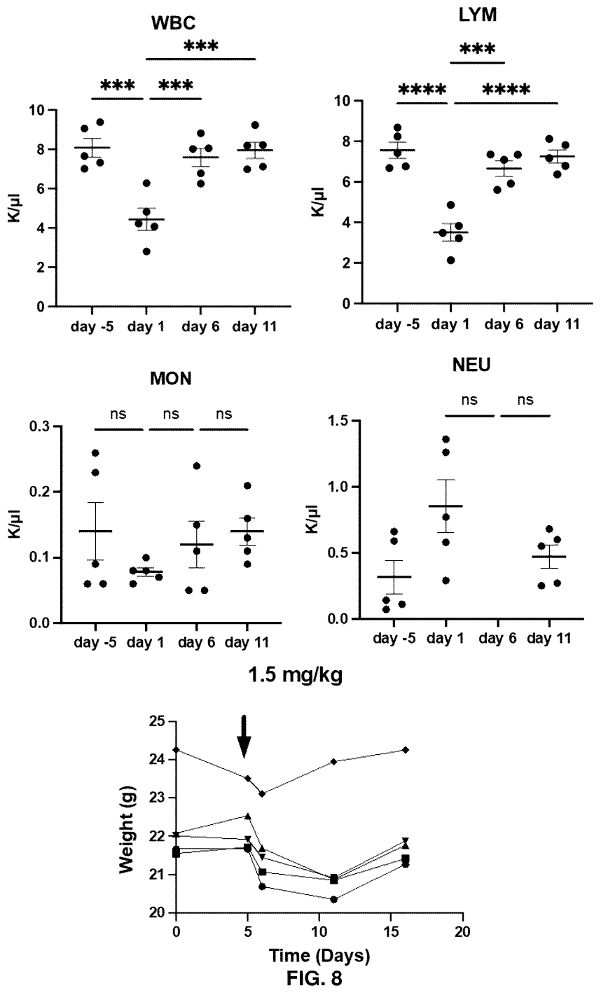

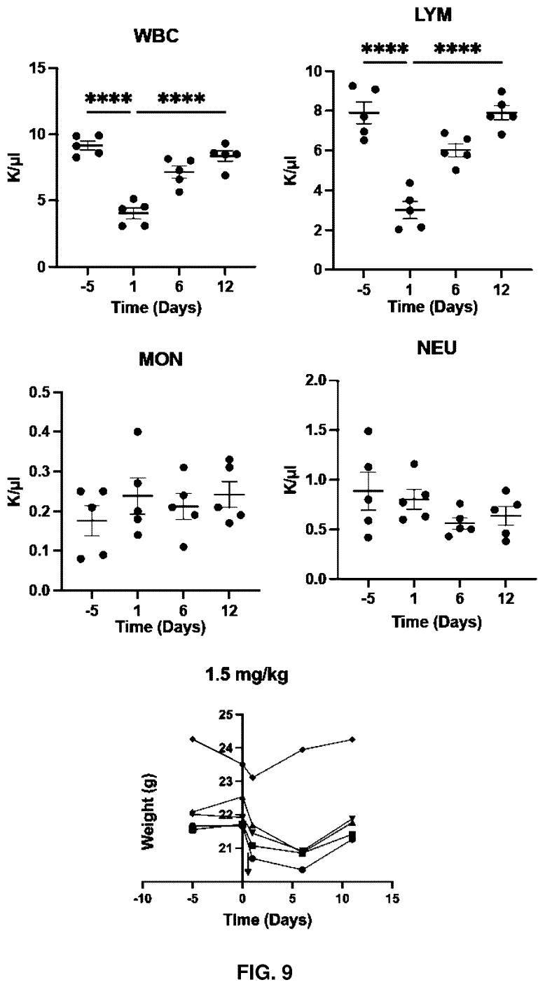

Effect of DTCCL8 in white blood cells (WBC) in mice. Immune cells are responsive to CCL8 because they express several receptors for CCL8 such as CCR2, CCR3, CCR5 and others, and thus, they are predicted to be sensitive to DTCCL8. Therefore, we explored the effects of DTCCCL8 in the blood profile of wild type mice. To that end, mice received 1.5 mg/kg DTCCL8 i.p., and their WBCs were assessed 5 days prior and 1, 6, and 11 days after the administration of the cytotoxic compound. The dose of DTCCL8 was determined by pilot experiments and was determined to be well-tolerated. As shown in FIG. 8, 1 day after the administration of DTCCL8, total white blood cell count was significantly decreased, but rebounded after 12 days. This decrease was primarily due to a decrease in lymphocytes (FIG. 8) which are known to express CCR5 (Loetscher et al, 1998). The effect of CCL8 in monocytes and neutrophils was insignificant at this dose (Loetscher et al, 1998). Body weight changes were insignificant after DTCCL8 administration. To confirm these findings, the treatment experiment was repeated independently, and a similar trend in WBC, lymphocyte, monocyte, and neutrophil counts was observed (FIG. 9).

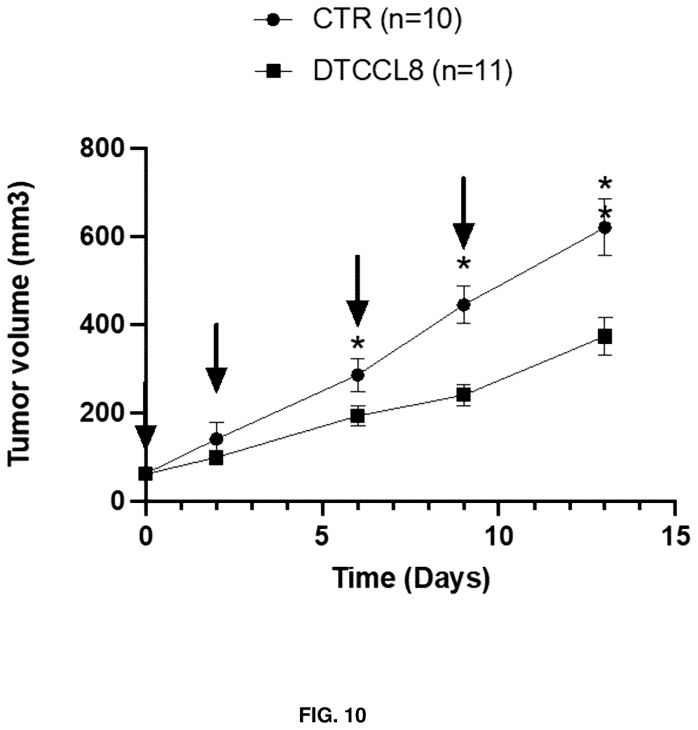

DTCCL8 inhibits breast cancer growth in mice. Subsequently, we sought to evaluate the potential anticancer activity of DTCCL8 in breast cancers at which the effect of CCL8 is established. Since both cancer cells and cells of the microenvironment are responsive to CCL8 we sought to explore the effects of DTCCL8 in mouse breast cancer in mice that possess intact immune system, as opposed to human breast cancer xenografts at which the host's immune system is compromised. To that end, we utilized isografts of primary mouse breast cancers triggered by polyoma middle T oncogene, that were transplanted in wild type isogenic mouse hosts. These are highly aggressive tumors that double in size every 4-5 days. Mice were treated when tumors became measurable, at about 50-70 mm³. Treatment involved i.p. administration of DTCCL8 at 1.5 mg/kg at days 0, 6 and 9, that is well tolerated by the mice. As shown in FIG. 10, 9 days after initiation of therapy, tumor size decreased significantly in the DTCCL8 group compared to controls.

### Discussion

We report the development and preliminary in vitro and in vivo characterization of DTCCL8, a cytotoxic peptide conjugate comprising diphtheria toxin (DT) linked to the chemokine hCCL8. The development of this conjugate was prompted by the observation that CCL8 receptors, like other chemokine receptors, are overexpressed in several cancers including breast cancers (Thomas et al, 2019). However, their redundancy in expression profile, ligand specificity, and function, reduces their potential utility in clinical practice (Bromley et al, 2020). The same redundancy applies, not only to the receptors but to their ligands as well, for which neutralization of a single target would likely not provide clinically relevant results. Furthermore, the expression of these chemokine receptors is not limited to the cancer cells but to the cells of the tumor stroma, providing a promising therapeutic target (Müller et al, 2001).

We hypothesized that this redundancy in expression of ligands and receptors, instead of a limitation could be converted to an advantage, enabling targeting of cytotoxic agents to tumors irrespectively of the specific receptor signature, for as long as responsiveness to CCL8 is retained. To that end we developed DTCCL8 which exhibits specific toxicity to cancer cells by a manner that depends on CCR5 and likely other CCL8 receptors' expression. This conclusion is further supported by our observation that antibody-mediated CCR5 blockade significantly reduced DTCCL8-induced cytotoxicity in both MDA-MB-231 and BT549 cells, while DT386 activity remained unaffected. These results confirm that DTCCL8 selectively targets cells through receptor engagement rather than nonspecific uptake. The cytotoxicity of DT386 was likely due to its internalization via non-canonical mechanisms, as previously demonstrated for the structurally similar DT385 (Zhang et al., 2010). DTCCL8 was effective in inhibiting the growth of highly aggressive mouse breast cancer isografts, triggered by the middle T oncogene of polyoma virus. DTCCL8, at a dose that was effective against breast cancers, was not toxic in mice. As expected, transiently, a reduction of WBCs, due to lymphocyte decrease, was noted after DTCCL8 administration, but the effect was subsequently abolished.

The specific targeting of immune cells suggests that DTCCL8, besides cancers that are responsive to CCL8, may also be effective against other conditions that involve aberrant immune response activation. These include ARDS and GVHD, at which a massive accumulation of immune cells occurs, and both have been linked to supraphysiological levels of CCL8 expression (Igarashi et al, 2014; Hori et al, 2008; Blanco-Melo et al, 2020; Suhre et al, 2021).

DTCCL8 represents a first of its class anticancer agent that targets tumors based on their capacity to respond to a specific chemokine such as CCL8. It is plausible that modulation, namely upregulation of CCL8 activity, in combination with other chemokine-cytotoxic drug combinations, may provide a novel strategy for the management of breast and other cancers, depending on their specific responsiveness to specific chemokines. It is noted that the potential availability of such cytotoxic peptides, in combination with the ability to rapidly evaluate the responsiveness of primary tumors to these drugs by ex vivo screening, will provide personalized strategies for the management of cancers exhibiting high deregulation in chemokine networks.

### Methods

### Design and Construction of Fusion Toxin Vectors

A custom plasmid containing the human CCL8 fusion toxin construct (DT386-hCCL8-6×His) was synthesized by GenScript and cloned into the pET30a (+) backbone between the NdeI and HindIII restriction sites (SEQ ID NO:4) (NdeI-HisTag-DT386-(Gly₄Ser)₂-hCCL8-HindIII). The encoded fusion (SEQ ID NO:5) (HisTag-DT386-(Gly₄Ser)₂-hCCL8) (FIG. 1A, top panel) includes the first 386 amino acids of the diphtheria toxin (SEQ ID NO:1) connected to hCCL8 (SEQ ID NO:2) by a (G₄S) 2 (SEQ ID NO:3). Six histidines (6×His tag) were added at the N-terminus for purification purposes. The sequence of the construct was verified by Sanger sequencing. To produce a truncated diphtheria toxin, we used splicing overlap extension polymerase chain reaction (SOEingPCR) with the following primers: Forward, 5′-CATATGCACCATCACCACCA-3′ (SEQ ID NO:6) containing NdeI cut site and reverse, 5′-AAGCTTTTAGCCGGTTTTATGACC-3′ (SEQ ID NO:7) containing HindIII cut site. This fragment was then cloned into a second plasmid, from which the original construct was removed by digestion with NdeI and HindIII. The new construct containing only the DT386 fragment was then cloned into the pET30a (+) backbone using these restriction sites, yielding a truncated diphtheria construct.

### Expression and Purification of Fusion Toxins

Both plasmids were transformed into BL21 (DE3) E. coli cells independently. Positive clones were observed in LB (Luria-Bertani) agar plates with 50 μg/mL kanamycin. A single colony was picked and transferred to 4 mL LB media with 50 μg/mL kanamycin. The starter culture was grown at 37° C. overnight at 250 RPM. The starter culture was then expanded in 1 liter of LB (Luria-Bertani) medium (supplemented with kanamycin at 50 ug/mL) by incubating at 37° C. and 250 RPM until the OD₆₀₀ reached 0.7. Protein expression was induced with 0.3 mM isopropyl b-D-1-thiogalactopyranoside (IPTG) and further cultured for 3 h at 37° C. Cells were harvested by centrifugation, and the expression of DT386-CCL8 in total cell protein from uninduced and induced samples were analyzed by SDS-Polyacrylamide gel electrophoresis (SDS-PAGE). Cell pellet was lysed using B-PER Bacterial Protein Extraction Reagent (Pierce), 0.2 mg/mL lysozyme (ThermoFisher), 1× Halt Protease Inhibitor Cocktail (ThermoFisher) and DNase I (Invitrogen). Inclusion bodies were dissolved using Inclusion Body Solubilization Reagent (Pierce) and 1 mM dithiothreitol (DTT). The protein refolding method was adapted from the protocol described by Moghadam et al. 2015. Briefly, the solubilized inclusion bodies were added to a refolding buffer containing 50 mM Tris-HCl, pH 8.5, 0.4 M sucrose, 10% glycerol, 0.5% Triton X-100, 0.3 mM glutathione disulfide (GSSG), and 3 mM glutathione (GSH) at a rate of 500 μL/hour while being stirred at 200 RPM at 4° C. The refolding solution was then stirred for 72 hours at 4° C. The solution was centrifuged at 9000×g for 20 min at 4° C. and supernatant was collected for purification. The His-tagged proteins were purified using Ni-NTA affinity chromatography independently. The supernatant fraction of the refolded protein was supplemented with sodium chloride to a final concentration of 1.5 M and imidazole to 5 mM. The refolded solution was then applied to Ni-NTA resin (GenScript) pre-equilibrated with binding buffer (50 mM Tris-HCl, pH 8.0, 300 mM NaCl, 10 mM imidazole). After incubation for 30 minutes at 4° C., the resin was washed with 20 column volumes of wash buffer (50 mM Tris-HCl, pH 8.0, 300 mM NaCl, 20 mM imidazole). The bound protein was eluted with elution buffer (50 mM Tris-HCl, pH 8.0, 300 mM NaCl, 250 mM imidazole).

### SDS-PAGE and Western Blot

The purity and identity of the protein were assessed by SDS-PAGE and Western blot analysis. For SDS-PAGE, protein samples were mixed with Laemmli buffer and heated at 95° C. for 5 minutes before loading onto a 12% polyacrylamide gel. Following electrophoresis, the gel was stained with Coomassie Brilliant Blue to visualize protein bands. The fractions containing the desired protein were pooled and subjected to buffer exchange into PBS (pH 7.4) using Pierce Protein Concentrators PES (Pierce) with a 10K molecular weight cut-off (MWCO). Following buffer exchange, the pooled protein fractions were processed to remove endotoxins using Pierce High-Capacity Endotoxin Removal Spin Columns (Pierce) according to the manufacturer's instructions. The protein concentration was then determined using a BCA assay. For Western blotting, proteins were transferred from the gel onto a PVDF membrane. Two separate Western blots were performed. The first blot was probed with Mouse 6×-His Tag mAb HRP (MA1-21315-HRP) to confirm the presence of the His-tagged protein. The second blot was probed with a chimeric antibody targeting human CCL8, followed by a mouse anti-Human IgG1 Fc Secondary Antibody, HRP (a10648). Detection was carried out using an enhanced chemiluminescence (ECL) substrate.

### Cell Culture

HEK293T (RRID: CVCL_0045), MDA-MB-231 (RRID: CVCL_0062, TNBC), BT-474 (RRID: CVCL_0179, HER2+, ER+, PR+), BT-549 (RRID: CVCL_1092, TNBC) and MCF-7 (RRID: CVCL 0031, HER2−, ER+, PR+) cells were cultured in Dulbecco's Modified Eagle Medium (DMEM; Gibco) supplemented with 10% fetal bovine serum (FBS; Gibco) and 1% penicillin-streptomycin (Pen/Strep; Gibco). CHO-K1 cells were cultured in Ham's F-12 Nutrient Mixture (F-12; Gibco) with the same supplements. Cell lines were originally obtained by ATCC and thereafter maintained in our laboratory. All cell lines were maintained at 37° C. in a humidified atmosphere with 5% CO₂. All experiments were performed with mycoplasma-free cells. Human cell lines have been validated by standard STR analysis by the Functional genomics Core of the Center for Targeted Therapeutics.

Cytotoxicity Studies with DTCCL8 and DT

To evaluate the cytotoxicity of DTCCL8 and DT, in vitro experiments were conducted using CHO-K1, MDA-MB-231, MCF-7, BT-474, BT-549 and HEK293T cell lines. Cells were seeded in 96-well plates at a density of 7,000 cells per well in triplicate. The cells were allowed to adhere overnight and then treated with a series of nine concentrations of each toxin variant, starting at 2500 nM and using serial dilutions to achieve final concentrations of 1250, 625, 312.3, 156.3, 78.1, 39, 19.5, and 9.7 nM. The treatments were applied for 72 hours. Following the incubation period, cell viability was assessed using the XTT assay. The XTT reagent was added to each well according to the manufacturer's instructions, and the plates were incubated for an additional 4 hours at 37° C. The absorbance was then measured at 450 nm with a reference wavelength of 650 nm using a microplate reader.

Transient Transfection with Human CCR5 Receptor

CHO-K1, and MDA-MB-231 cells were transiently transfected with a plasmid encoding the human CCR5 receptor (pcDNA3-CCR5, a gift from Erik Procko; Addgene plasmid #98943; n2t.net/addgene: 98943; RRID: Addgene_98943). Cells were seeded in 96-well plates at a density of 7,000 cells per well in triplicate and allowed to adhere overnight. The next day, transfection complexes were prepared using Lipofectamine 3000 (Invitrogen) according to the manufacturer's protocol. For each well, the CCR5 plasmid DNA was mixed with Lipofectamine 3000 reagent in Opti-MEM (Gibco) and incubated for 15 minutes at room temperature to form DNA-Lipofectamine complexes. The transfection complexes were then added to the cells, and the plates were incubated at 37° C. in a 5% CO₂ atmosphere for 24 hours to allow for transgene expression. After the transfection period, cells were treated with varying concentrations of the toxin for 72 hours. Following the treatment, cell viability was assessed using the XTT assay.

CCR5 Blockade with Neutralizing Antibody

MDA-MB-231 and BT549 cells were seeded at 7,000 cells per well in 96-well plates (triplicates per condition) and allowed to adhere overnight. Cells were pre-treated with a CCR5-blocking antibody (clone 45531, R&D Systems) at 10 μg/mL for 1 hour prior to toxin exposure. Following pre-treatment, cells were treated with 500 nM of DTCCL8 or DT386 while maintaining the antibody in the media at a concentration of 7.5 μg/mL throughout the 7-day experiment. Control wells included untreated cells, cells treated with antibody alone, and cells treated with toxin without antibody. Media containing antibody and toxin was replenished on day 3. On day 7, cell viability was quantified using the XTT assay. Results were normalized to untreated controls.

### Fluorescent Labeling and Internalization Assay

DTCCL8 and DT386 were fluorescently labeled using DyLight™ 633 NHS Ester (Thermo Fisher Scientific), which covalently binds to primary amines (typically lysine residues) on the protein. The labeling reactions were carried out according to the manufacturer's protocol, and Pierce™ Dye Removal Columns (Thermo Fisher Scientific) were used to eliminate excess unbound dye. MDA-MB-231 and BT549 cells were seeded in 96-well glass-bottom plates (P96-1.5H-N, Cellvis) pre-coated with Poly-D-Lysine (A3890401, Thermo Fisher Scientific) at a density of 10,000 cells per well in triplicate and incubated overnight. Cells were treated for 24 hours with 50 nM or 25 nM of DyLight 633-labeled DTCCL8 or DT386. For competition conditions, the labeled DTCCL8 peptide was co-incubated with a tenfold molar excess of either a chimeric anti-CCL8 antibody (VL1B) or a human IgG1 isotype control (500 nM or 250 nM, respectively). Imaging was performed using a Zeiss LSM 700 confocal microscope equipped with a 10× objective. For each well, three representative fields were captured. Fluorescence intensity was quantified using ImageJ by measuring the total cell intensity around automatically detected nuclei using a custom thresholding algorithm, with manual validation of nuclei segmentation quality. Quantified values were normalized to control-treated wells.

### Tumor Growth Studies

Mice were originally obtained from Jackson Labs and subsequently maintained in our facilities. Wild type female C57BL/6 mice, aged 2-4 months (JAX stock #000664), were used for the in vivo analysis. Mammary tumors from B6.FVB-Tg (MMTV-PyMT; JAX stock #022974, Davie S A et al. 2007) mice backcrossed into C57BL/6 background, were harvested and implanted into the flanks of 21 recipient mice using a trocar needle. A single tumor was used for all implantations to ensure consistency. Tumor growth was monitored, and once the tumors reached a similar size across all mice, the animals were randomly assigned into two groups: a control group (n=10) and a treatment group (n=11). The treatment group received 1.5 mg/kg of DTCCL8 administered intraperitoneally every two days, while the control group received an equivalent volume of vehicle solution. Tumor sizes were measured using calipers were recorded twice a week. The volume of the tumors was calculated using the formula: V=0.5×L×W2. Mice were observed for signs of distress or adverse reactions throughout the study period. Tumor size and weight were meticulously recorded to assess the efficacy of the DTCCL8 treatment compared to the control.

### Blood Collection and Analysis

Blood samples were collected from female C57BL/6 mice aged 2-4 months at specified time points for two treatment groups. For the first group, blood was collected 5 days before treatment, and 1, 6, and 11 days after treatment with DTCCL8 at a dosage of 1.5 mg/kg. Blood was drawn from the submandibular vein using a sterile lancet and collected into EDTA-coated microcentrifuge tubes to prevent clotting. On each collection day, mice were gently restrained, and the puncture site was cleaned with an alcohol swab. Using a sterile lancet, a small puncture was made in the submandibular vein and approximately 50-100 μL of blood was collected per mouse. After blood collection, gentle pressure was applied to the puncture site with sterile gauze to stop any bleeding. The collected blood samples were gently mixed by inversion to ensure proper anticoagulation. Hematological parameters were measured using the Vetscan HM5 hematology analyzer (Abaxis), following the manufacturer's instructions.

### REFERENCES

Alkharabsheh, O.; Frankel, A. E. Clinical Activity and Tolerability of SL-401 (Tagraxofusp): Recombinant Diphtheria Toxin and Interleukin-3 in Hematologic Malignancies. Biomedicines 2019, 7, 6.
Abdul-Rahman, T., Ghosh, S., Badar, S. M. et al. The paradoxical role of cytokines and chemokines at the tumor microenvironment: a comprehensive review. Eur J Med Res 29, 124 (2024).
Barbai T., Fejös Z., Puskas L. G., Tímár J., Rásó E. The importance of microenvironment: the role of CCL8 in metastasis formation of melanoma. Oncotarget. 2015; 6:29111-29128.
Blanco-Melo D, Nilsson-Payant B E, Liu W C, Uhl S, Hoagland D, Møller R, Jordan T X, Oishi K, Panis M, Sachs D, Wang T T, Schwartz R E, Lim J K, Albrecht R A, tenOever B R. Imbalanced Host Response to SARS-COV-2 Drives Development of COVID-19. Cell. 2020 May 28; 181 (5): 1036-1045.e9.
Blaszczyk J, Coillie E V, Proost P, Damme J V, Opdenakker G, Bujacz G D, Wang J M, Ji X. Complete crystal structure of monocyte chemotactic protein-2, a C C chemokine that interacts with multiple receptors. Biochemistry. 2000; 39 (46): 14075-81.
Bromley, S. K., Thomas, S. Y., & Luster, A. D. (2020). Understanding the mechanisms that facilitate specificity, not redundancy, of chemokine-mediated immune cell recruitment. Immunological Reviews, 289 (1), 9-31.
Cassetta L., Fragkogianni S., Sims A. H., Swierczak A., Forrester L. M., Zhang H., Soong D. Y. H., Cotechini T., Anur P., Lin E. Y., et al. Human tumor-associated macrophage and monocyte transcriptional landscapes reveal cancer-specific reprogramming, biomarkers, and therapeutic targets. Cancer Cell. 2019; 35:588-602.e10
Davie, S. A., Maglione, J. E., Manner, C. K., Young, D., Cardiff, R. D., MacLeod, C. L., & Ellies, L. G. Effects of FVB/N J and C57B L/6J strain backgrounds on mammary tumor phenotype in inducible nitric oxide synthase deficient mice. Transgenic Research, 2007.
Farmaki E., Chatzistamou I., Kaza V., Kiaris H. A CCL8 gradient drives breast cancer cell dissemination. Oncogene. 2016; 35:6309-6318.
Fang, W. B., Sofia Acevedo, D., Smart, C. et al. Expression of CCL2/CCR2 signaling proteins in breast carcinoma cells is associated with invasive progression. Sci Rep 11, 8708 (2021).
Fang, W. B., Jokar, I., Zou, A., Lambert, D., Dendukuri, P. and Cheng, N. (2012). CCL2/CCR2 chemokine signaling coordinates survival and motility of breast cancer cells through Smad3 protein- and p42/44 Mitogen-activated Protein Kinase (MAPK)-dependent mechanisms. J. Biol. Chem. 287, 36593-36608.
Farmaki E., Kaza V., Chatzistamou I., Kiaris H. CCL8 promotes postpartum breast cancer by recruiting M2 macrophages. iScience. 2020; 23:101217.
Heredia J D, Park J, Brubaker R J, Szymanski S K, Gill K S, Procko E. J. Mapping Interaction Sites on Human Chemokine Receptors by Deep Mutational Scanning. Immunol. 2018 Apr. 20. pii: jimmunol.1800343.
Hughes C E, Nibbs R J B. A guide to chemokines and their receptors. FEBS J. 2018 August; 285 (16): 2944-2971.
Hori T., Naishiro Y., Sohma H., Suzuki N., Hatakeyama N., Yamamoto M., Sonoda T., Mizue Y., Imai K., Tsutsumi H., Kokai Y. CCL8 is a potential molecular candidate for the diagnosis of graft-versus-host disease. Blood. 2008; 111:4403-4412.
Igarashi K., Takei N., Hori T., Yamamoto M., Kokai Y. CCL8 deficiency in host strongly inhibits early mortality and morbidity of graft-versus-host disease in mice. Blood. 2014; 124:1096.
Igarashi K, Hori T, Yamamoto M, Sohma H, Suzuki N, Tsutsumi H, Kawasaki Y, Kokai Y. CCL8 deficiency in the host abrogates early mortality of acute graft-versus-host disease in mice with dysregulated IL-6 expression. Exp Hematol. 2022 February; 106:47-57. doi: 10.1016/j.exphem.2021.11.006. Epub 2021 Nov. 20. Erratum in: Exp Hematol. 2023 November; 127:70.
Jiao X, Velasco-Velazquez M A, Wang M, Li Z, Rui H, Peck A R, et al. CCR5 governs DNA damage repair and breast cancer stem cell expansion. Cancer Res. 2018; 78 (7): 1657-71.
Liu H, Liu Z, Chen J, Chen L, He X, Zheng R, Yang H, Song P, Weng D, Hu H, Fan L, Xiao H, Kaufmann S H, Ernst J, Ge B. Induction of CCL8/MCP-2 by mycobacteria through the activation of TLR2/PI3K/Akt signaling pathway. PLOS One. 2013; 8 (2): e56815.
Loetscher, P., Uguccioni, M., Bordoli, L. et al. CCR5 is characteristic of Th1 lymphocytes. Nature 391, 344-345 (1998).
Lou X, Zhao K, Xu J, Shuai L, Niu H, Cao Z, Wang J, Zhang Y. CCL8 as a promising prognostic factor in diffuse large B-cell lymphoma via M2 macrophage interactions: A bioinformatic analysis of the tumor microenvironment. Front Immunol. 2022 Aug. 22; 13:950213.
Moghadam M, Ganji A, Varasteh A, Falak R, Sankian M. Refolding process of cysteine-rich proteins: Chitinase as a model. Rep Biochem Mol Biol. 2015 October; 4 (1): 19-24.
Müller, A., Homey, B., Soto, H., Ge, N., Catron, D., Buchanan, M. E., . . . & Lipp, M. (2001). Involvement of chemokine receptors in breast cancer metastasis. Nature, 410 (6824), 50-56.
Naderi A, Farmaki E, Chavez B, Cai C, Kaza V, Zhang Y, Soltanmohammadi E, Daneshvar N, Chatzistamou I, & Kiaris H. Beneficial effects of CCL8 inhibition at LPS-induced lung injury. iScience. 2022.
Nagarsheth, N., Wicha, M. & Zou, W. Chemokines in the cancer microenvironment and their relevance in cancer immunotherapy. Nat Rev Immunol 17, 559-572 (2017).
Ozga A J, Chow M T, Luster A D. Chemokines and the immune response to cancer. Immunity. 2021 May 11; 54 (5): 859-874. doi: 10.1016/j.immuni.2021.01.012. Epub 2021 Apr. 10.
Severa M, Islam S A, Waggoner S N, Jiang Z, Kim N D, Ryan G, Kurt-Jones E, Charo I, Caffrey D R, Boyartchuk V L, Luster A D, Fitzgerald K A. The transcriptional repressor BLIMP1 curbs host defenses by suppressing expression of the chemokine CCL8. J Immunol. 2014 Mar. 1; 192 (5): 2291-304.
Shafiee F, Aucoin M G, Jahanian-Najafabadi A. Targeted Diphtheria Toxin-Based Therapy: A Review Article. Front Microbiol. 2019 Oct. 18; 10:2340.
Sokol C L, Camire R B, Jones M C, Luster A D. The Chemokine Receptor CCR8 Promotes the Migration of Dendritic Cells into the Lymph Node Parenchyma to Initiate the Allergic Immune Response. Immunity. 2018 Sep. 18; 49 (3): 449-463.e6.
Suhre K, Sarwath H, Engelke R, Sohail M U, Cho S J, Whalen W, Alvarez-Mulett S, Krumsiek J, Choi A M K, Schmidt F. Identification of Robust Protein Associations With COVID-19 Disease Based on Five Clinical Studies. Front Immunol. 2022 Jan. 25; 12:781100.
Syed Y Y. Tagraxofusp: First Global Approval. Drugs. 2019 April; 79 (5): 579-583.
Thoutam A, Breitzig M, Lockey R, Kolliputi N. Coronavirus: a shift in focus away from IFN response and towards other inflammatory targets. J Cell Commun Signal. 2020 December; 14 (4): 469-470.
Thomas J. K., Mir H., Kapur N., Bae S., Singh S. C C chemokines are differentially expressed in Breast Cancer and are associated with disparity in overall survival. Sci. Rep. 2019; 9:4014-4112.
Velasco-Velazquez, M. & Pestell, R. G. The CCL5/CCR5 axis promotes metastasis in basal breast cancer. Oncoimmunology 2, e23660.
Wang M, Ge B, Li R, Wang X, Lao J, Huang F. Milligram production and biological activity characterization of the human chemokine receptor CCR3. PLOS One. 2013; 8 (6): e65500.
Wang M Q, Ge B S, Yang Q X, Jiang X Y, Huang F. High-level production of biologically active chemokines in Escherichia Coli. Process Biochem. 2014; 49 (4): 706-14.
Wang X, Han Y, Peng J, He J. CCR5 is a prognostic biomarker and an immune regulator for triple negative breast cancer. Aging (Albany N Y). 2021 Oct. 30; 13 (20): 23810-23830.
Wang, H., Wang, Z., Zhang, H., Qi, Z., Johnson, A. C., Mathes, D., Pomfret, E. A., Rubin, E., Huang, C. A. and Wang, Z. (2020), Bispecific human IL2-CCR4 immunotoxin targets human cutaneous T-cell lymphoma. Mol Oncol, 14:991-1000.
Zhang Y, Hollenbeck S T, Demeure M J, Hajighasemi M, Boddipalli L, Herlyn M, Plautz G E. Truncated Diphtheria Toxin (DT385) induces apoptosis in tumor cells without receptor binding. PLOS One. 2010 Jun. 8; 5 (6): e10498.
Zhang, X., Chen, L., Dang, Wq. et al. CCL8 secreted by tumor-associated macrophages promotes invasion and stemness of glioblastoma cells via ERK1/2 signaling. Lab Invest 100, 619-629 (2020).
Zheng, Q., Wang, Z., Zhang, H., Huang, Q., Madsen, J. C., Sachs, D. H., Huang, C. A. and Wang, Z. (2017), Diphtheria toxin-based anti-human CD19 immunotoxin for targeting human CD19+ tumors. Mol Oncol, 11:584-594.

SEQUENCES

DT386
GADDVVDSSKSFVMENFSSYHGTKPGYVDSIQKGIQKPKSGTQGNYDDD
WKGFYSTDNKYDAAGYSVDNENPLSGKAGGVVKVTYPGLTKVLALKVD
NAETIKKELGLSLTEPLMEQVGTEEFIKRFGDGASRVVLSLPFAEGSSSVE
YINNWEQAKALSVELEINFETRGKRGQDAMYEYMAQACAGNRVRRSVG
SSLSCINLDWDVIRDKTKTKIESLKEHGPIKNKMSESPNKTVSEEKAKQYL
EEFHQTALEHPELSELKTVTGTNPVFAGANYAAWAVNVAQVIDSETADN
LEKTTAALSILPGIGSVMGIADGAVHHNTEEIVAQSIALSSLMVAQAIPLV
GELVDIGFAAYNFVESIINLFQVVHNSYNRPAYSPGHKT (SEQ ID NO: 1)
hCCL8
QPDSVSIPITCCFNVINRKIPIQRLESYTRITNIQCPKEAVIFKTQRGKEVCA
DPKERWVRDSMKHLDQIFQNLKP (SEQ ID NO: 2)
(Gly₄Ser)₂ Linker
GGGGSGGGGS (SEQ ID NO: 3)
NdeI - HisTag - DT386 - (Gly₄Ser)₂ - hCCL8 - HindIII
catatgcaccatcaccaccaccatggagctgatgacgtggttgattctagcaaatcttttgtaatggaaaatttctcctcc
taccatggcacgaagccgggttatgttgactcaatccagaaaggcatccaaaagccgaagagcggcacgcagggt
aactacgacgacgactggaagggcttctatagcaccgataacaagtacgatgctgcgggttactcggtggacaacg
aaaacccgctgtccggcaaggcgggtggtgtcgtcaaagtcacctacccgggtctgaccaaggtgttggccctgaa
agtagacaatgcggaaaccatcaaaaaagaactgggtctgtcgttaaccgaaccgttgatggagcaagtgggtactg
aggaattcatcaagcgctttggcgatggtgccagccgtgtggtcctgtcattgccattcgcagagggctcgtcgtccgt
tgagtatattaacaactgggaacaggcaaaagcgcttagcgtggagttagagatcaactttgaaacacgtggtaagc
gtggacaggatgcaatgtatgaatacatggcacaagcgtgtgctggtaatagagtgcgtcgcagcgtgggtagctct
ctgagctgcattaacctggattgggatgttattcgtgataaaacaaagacgaagattgaatctctaaaggagcacggtc
cgatcaaaaataagatgagcgaatccccgaataagaccgtgtccgaagagaaggcgaaacaatacctggaggagt
tccaccagaccgctttggagcacccggagctctctgagctgaaaaccgttaccggcaccaatccggtttttgcaggc
gcgaattacgcggcgtgggcagtcaacgttgctcaggtgattgatagcgagactgcggacaacctggagaaaacg
accgcggctctctccatcctgccgggcatcggcagcgttatgggtattgccgacggcgcggttcaccataataccga
agaaattgtggctcaaagcattgcgttgtctagcctgatggttgcgcaagcaattccactggttggcgaattggttgaca
tcggctttgccgcgtataacttcgtggagagcatcattaacctgttccaagttgtgcacaacagctataaccgccctgca
tatagcccgggtcataaaaccggcggtggtggtagtggtggaggcggcagccagccggattctgtgagcatcccg
atcacctgctgctttaacgtgatcaatcgtaaaatcccgattcaacgtctggagagttacacgcgtattactaacatcca
gtgcccgaaggaggcggtgatcttcaaaacccagcgtggcaaagaagtttgtgctgacccgaaagaacgctgggtt
cgcgactccatgaagcatctggaccagattttccagaatctgaaaccgtaatgaaagctt (SEQ ID NO: 4)
HisTag - DT386 - (Gly₄Ser)₂ - hCCL8
MHHHHHHGADDVVDSSKSFVMENFSSYHGTKPGYVDSIQKGIQKPKSGT
QGNYDDDWKGFYSTDNKYDAAGYSVDNENPLSGKAGGVVKVTYPGLT
KVLALKVDNAETIKKELGLSLTEPLMEQVGTEEFIKRFGDGASRVVLSLPF
AEGSSSVEYINNWEQAKALSVELEINFETRGKRGQDAMYEYMAQACAG
NRVRRSVGSSLSCINLDWDVIRDKTKTKIESLKEHGPIKNKMSESPNKTVS
EEKAKQYLEEFHQTALEHPELSELKTVTGTNPVFAGANYAAWAVNVAQ
VIDSETADNLEKTTAALSILPGIGSVMGIADGAVHHNTEEIVAQSIALSSLM
VAQAIPLVGELVDIGFAAYNFVESIINLFQVVHNSYNRPAYSPGHKTGGG
GSGGGGSQPDSVSIPITCCFNVINRKIPIQRLESYTRITNIQCPKEAVIFKTQR
GKEVCADPKERWVRDSMKHLDQIFQNLKP (SEQ ID NO: 5)

What is claimed is:

1. A peptide construct comprising a cytotoxin linked to a CCL8 peptide.

2. The peptide construct of claim 1, wherein the cytotoxin comprises a cytotoxic peptide.

3. The peptide construct of claim 1, wherein the cytotoxin comprises a cytotoxic peptide and the cytotoxic peptide comprises a diphtheria toxin peptide.

4. The peptide construct of claim 1, wherein the cytotoxin comprises a cytotoxic peptide and the cytotoxic peptide comprises an amino acid sequence at least 80%, SEQ ID NO:1.

5. The peptide construct of claim 1, wherein the cytotoxin comprises a cytotoxic peptide and the cytotoxic peptide comprises an amino acid sequence at least 95% identical to SEQ ID NO: 1.

6. The peptide construct of claim 1, wherein the CCL8 peptide comprises an amino acid sequence at least 80% identical to SEQ ID NO:2.

7. The peptide construct of claim 1, wherein the CCL8 peptide comprises an amino acid sequence at least 95% identical to SEQ ID NO:2.

8. The peptide construct of claim 1, wherein the cytotoxin comprises a cytotoxic peptide and the cytotoxic peptide is linked to the CCL8 peptide via a peptide linker.

9. The peptide construct of claim 8, wherein the peptide linker has a length from 1 to 30 amino acids.

10. The peptide construct of claim 1, wherein the cytotoxin comprises a cytotoxic peptide and a C-terminus of the cytotoxic peptide is linked to an N-terminus of the CCL8 peptide.

11. The peptide construct of claim 1, wherein:
the cytotoxin comprises a cytotoxic peptide;
the cytotoxic peptide comprises an amino acid sequence at least 95% identical to SEQ ID NO: 1;
the CCL8 peptide comprises an amino acid sequence at least 95% identical to SEQ ID NO: 2; and
the cytotoxic peptide is linked to the CCL8 peptide via a peptide linker.

12. The peptide construct of claim 11, wherein a C-terminus of the cytotoxic peptide is linked to an N-terminus of the CCL8 peptide.

13. The peptide construct of claim 12, wherein the peptide linker has a length from 1 to 30 amino acids.

14. The peptide construct of claim 11, wherein the peptide linker has a length from 1 to 30 amino acids.

15. The peptide construct of claim 1, wherein:
the cytotoxin comprises a cytotoxic peptide;
the cytotoxic peptide comprises an amino acid sequence at least 99% identical to SEQ ID NO: 1;
the CCL8 peptide comprises an amino acid sequence at least 99% identical to SEQ ID NO: 2; and
the cytotoxic peptide is linked to the CCL8 peptide via a peptide linker having a length from 1 to 30 amino acids.

16. The peptide construct of claim 15, wherein a C-terminus of the cytotoxic peptide is linked to an N-terminus of the CCL8 peptide.

17. A method of treating a CCL8-related disease in a subject, the method comprising administering the peptide construct of claim 1 or a nucleic acid configured to express the peptide construct to the subject in an amount effective to treat the CCL8-related disease.

18. The method of claim 17, wherein the CCL8-related disease comprises one or more of cancer, graft versus host disease (GVHD), microbial infection, and pulmonary fibrosis acute respiratory distress syndrome (ARDS).

19. The method of claim 17, wherein the CCL8-related disease comprises cancer.

20. The method of claim 17, wherein the CCL8-related disease comprises breast cancer.


SEQUENCE LISTING (7 sequences)

SEQ ID NO: 1 - protein, 386 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKT

SEQ ID NO: 2 - protein, 76 aa, Homo sapiens
QPDSVSIPITCCFNVINRKIPIQRLESYTRITNIQCPKEAVIFKTQRGKEVCADPKERWVRDSMKHLDQIFQNLKP

SEQ ID NO: 3 - protein, 10 aa, synthetic construct
GGGGSGGGGS

SEQ ID NO: 4 - DNA, 1452 bp, synthetic construct
  source 1..1452: mol_type = other DNA
catatgcaccatcaccaccaccatggagctgatgacgtggttgattctagcaaatcttttgtaatggaaaatttctcctcctaccatggcacgaagccgggttatgttgactcaatccagaaaggcatccaaaagccgaagagcggcacgcagggtaactacgacgacgactggaagggcttctatagcaccgataacaagtacgatgctgcgggttactcggtggacaacgaaaacccgctgtccggcaaggcgggtggtgtcgtcaaagtcacctacccgggtctgaccaaggtgttggccctgaaagtagacaatgcggaaaccatcaaaaaagaactgggtctgtcgttaaccgaaccgttgatggagcaagtgggtactgaggaattcatcaagcgctttggcgatggtgccagccgtgtggtcctgtcattgccattcgcagagggctcgtcgtccgttgagtatattaacaactgggaacaggcaaaagcgcttagcgtggagttagagatcaactttgaaacacgtggtaagcgtggacaggatgcaatgtatgaatacatggcacaagcgtgtgctggtaatagagtgcgtcgcagcgtgggtagctctctgagctgcattaacctggattgggatgttattcgtgataaaacaaagacgaagattgaatctctaaaggagcacggtccgatcaaaaataagatgagcgaatccccgaataagaccgtgtccgaagagaaggcgaaacaatacctggaggagttccaccagaccgctttggagcacccggagctctctgagctgaaaaccgttaccggcaccaatccggtttttgcaggcgcgaattacgcggcgtgggcagtcaacgttgctcaggtgattgatagcgagactgcggacaacctggagaaaacgaccgcggctctctccatcctgccgggcatcggcagcgttatgggtattgccgacggcgcggttcaccataataccgaagaaattgtggctcaaagcattgcgttgtctagcctgatggttgcgcaagcaattccactggttggcgaattggttgacatcggctttgccgcgtataacttcgtggagagcatcattaacctgttccaagttgtgcacaacagctataaccgccctgcatatagcccgggtcataaaaccggcggtggtggtagtggtggaggcggcagccagccggattctgtgagcatcccgatcacctgctgctttaacgtgatcaatcgtaaaatcccgattcaacgtctggagagttacacgcgtattactaacatccagtgcccgaaggaggcggtgatcttcaaaacccagcgtggcaaagaagtttgtgctgacccgaaagaacgctgggttcgcgactccatgaagcatctggaccagattttccagaatctgaaaccgtaatgaaagctt

SEQ ID NO: 5 - protein, 479 aa, synthetic construct
MHHHHHHGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTGGGGSGGGGSQPDSVSIPITCCFNVINRKIPIQRLESYTRITNIQCPKEAVIFKTQRGKEVCADPKERWVRDSMKHLDQIFQNLKP

SEQ ID NO: 6 - DNA, 20 bp, synthetic construct
  source 1..20: mol_type = other DNA
catatgcaccatcaccacca

SEQ ID NO: 7 - DNA, 24 bp, synthetic construct
  source 1..24: mol_type = other DNA
aagcttttagccggttttatgacc